(19) United States
(12) Patent Application Publication
(10) Pub. No.: US 2004/0097534 A1
(43) Pub. Date: May 20, 2004

(54) Composition for the protection and regeneration of nerve cells containing berberine derivatives

(75) Inventors: Byung-Kil Choi, Seo-gu (KR); Yun-Hee Kim, Seoul (KR); Soo-Kyung Kim, Jung-gu (KR); Jung-Su Lim, Seoul (KR); Hyo-Sup Kim, Namdong-gu (KR); Dae-Sung Park, Seoul (KR); Chi-Young Chang, Bucheon-si (KR)

Correspondence Address: HESLIN ROTHENBERG FARLEY & MESITI PC, 5 COLUMBIA CIRCLE, ALBANY, NY, 12203, US

(73) Assignee: EUGENBIO INC., Chungcheongnam-do (KR)

(21) Appl. No.: 10/389,693

(22) Filed: Mar. 14, 2003

Related U.S. Application Data
(63) Continuation of application No. PCT/KR02/01307, filed on Jul. 10, 2002.

(30) Foreign Application Priority Data
Jul. 10, 2001 (KR) 2001-0041248
Jul. 10, 2002 (KR) 2002-0040015

(51) Int. Cl. A61K 31/4745

(52) U.S. Cl. 514/283

(57) ABSTRACT

Disclosed is a composition for protecting nerve cells, promoting nerve cell growth and regenerating nerve cells comprising berberine, derivatives thereof or pharmaceutically acceptable salts thereof. The composition has protective effects against apoptosis of neuronal stem cells and differentiated neuronal stem cells, an effect of inducing the regeneration of nerve cells, a regenerative effect on neurites, a neuroregenerative effect on central nerves and peripheral nerves, a reformation effect on neuromuscular junctions, and a protective effect against apoptosis of nerve cells and a neuroregenerative effect in animals suffering from dementia and brain ischemia. Therefore, the composition can be used as a therapeutic agent for the prevention and treatment of neurodegenerative diseases, ischemic nervous diseases or nerve injuries, and for the improvement of learning capability.

### CROSS REFERENCE TO RELATED APPLICATIONS

This application is a continuation of International Application PCT/KR02/01307, which was filed Jul. 10, 2002, claiming priority from Korean Patent Applications 2001-0041248, which was filed Jul. 10, 2001 and 2002-0040015, which was filed Jul. 10, 2002. The entire content of each of the prior applications is incorporated herein by reference.

### FIELD OF THE INVENTION

The present invention relates to a composition for protecting nerve cells, promoting nerve cell growth and regenerating nerve cells, or for preventing and treating nerve injuries or nervous diseases, comprising a compound of the following formula 1:

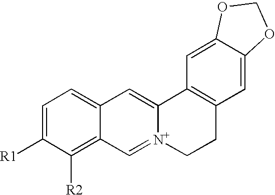

wherein

R1 and R2 are the same or different from each other and independently selected from the group consisting of alkoxy group, alkyl group, hydrogen atom, methylenedioxy group, substituted benzyl group, propoxy group, octyl group, alkenyl group, alkynyl group, amino group, amide group, cyano group, thiocyano group, aldehyde group and halogen atom, derivatives thereof, or pharmaceutically acceptable salts thereof.

### BACKGROUND OF THE INVENTION

Synapses are the connection points between nerve cells, and one nerve cell connects to 1000-5000 other nerve cells on average. It is estimated that since at least 10¹¹ nerve cells exist in the human brain, there are at least 10¹⁴ synapses in the human brain. All complex and various brain functions, for example thoughts, sensations, memory, learning and actions, cannot be understood without consideration of these neural networks.

Synaptic connections are essential to nerve cell survival. Special functions according to the connections between nerve cells make it possible to express high-level brain functions intrinsic to humans. In particular, it is known that once the central nervous system is damaged, its regeneration is very difficult. Many ideas and attempts for treating damaged nerve tissues or chronic degenerative diseases have been made in various ways.

In the 1940's, Hamburger and Levi-Montalcini discovered an unidentified substance indispensable for survival of motor neurons in the differentiation process of Chick embryo limb, and proposed the neurotrophic factor hypothesis. Based on the hypothesis, NGF (nerve growth factor) was first discovered, and discoveries of neurotrophic factors such as BDNF (brain-derived neurotrophic factor), NT-3 (neurotrophic factor-3), etc., followed. Further, it was found in some transgenic animal experiments which types of nerve growth factors are necessary for survival of each differentiated nerve cell population. Also, it was found that not only neurotrophins but also some cytokines are involved in nerve cell survival. When neurotrophins or cytokines are not supplied or receptors for these neurotrophins or cytokines are not expressed in the cells, nerve cells die.

There are two nerve cell death pathways, like all other cells: necrosis and apoptosis. Necrosis and apoptosis have different morphological and molecular biological characteristics. When an axon is cut (axotomy), a part attached to the cell body and a terminal forming a synapse are separated each other. Such axotomy leads to not only synaptic denaturation due-to cut off of supply of protein factors from target cell body, but also synaptic detachment. That is, regeneration is a key to nerve cell survival. Dead nerve cells are replaced with glial cells in the peripheral nervous system, and astrocytes or microglias in the central nervous system, in a process called “synaptic stripping”. In addition, immune system cells such as monocytes, macrophages, etc., can replace the dead nerve cells, depending on the extent of damages. Many theories explaining mechanisms of physical injuries to nerve cells, acute neurotoxicity, acute and chronic nervous disorders, dementia, epileptic., etc. have been introduced, but these theories all have a common point. That is, these diseases affect nerve cells and supporting tissue cells thereof. These cells extend horizontally and perpendicularly to form many dendrites and axons, which form many neural networks. Abnormalities in the neural nets lead to deregulation in signal transmission and cause various cranial nervous system diseases. The glutamatergic neural net responding to glutamate, an excitatory neurotransmitter, is a neural net to which has drawn attention in terms of development of acute and chronic cranial nervous diseases.

All mammalian brains develop a systematic neural network through a series of division, differentiation, survival and apoptosis of neuronal stem cells, and synaptic formation, thereby performing complex brain functions. In the adult brain, cranial nerve cells produce many substances necessary for nerve growth to make their axons and dendrites grow. Therefore, as new learning and memories are introduced, synaptic connections and neural networks are continuously remodeled. In the differentiation and synaptic formation of nerve cells, cells not receiving target-derived survival factors such as nerve growth factors die, and cell death due to stress and cytotoxic agents is a major cause of degenerative brain diseases. Unlike the central nervous system, when the peripheral nervous system is injured, the axons regrow but require a long time to do so. Axons in the distal stump from the injury sites, which are not connected to their cell bodies, undergo Wallerian degeneration. Their cell bodies undergo axonal regrowth, and Schwann cells are regenerated through a series of divisions and regulate the survival and apoptosis, and axonal regrowth of target nerves.

It was recently shown that neuronal stem cells exist in the adult brain. The development and differentiation of the stem cells in the adult brain lead to the regeneration of nerve cells (Johansson, C. B., Momma S., Clarke D. L., Risling M., Lendahl U., and Frisen J. (1999) Identification of a neural stem cell in the adult mammalian central nervous system, Cell 96, 25-34). Neuronal stem cells are mainly found in the subventricular zones of striatum adjacent to lateral ventricles. Neural stem cells in the subgranular zones at dentate gyrus of the hippocampus divide to form granule cells (van Praag, H., Schinder, A. F., Christie, B. R., Toni, N., Palmer, T. D. & Gage, F. H. (2002) Functional neurogenesis in the adult hippocampus. Nature 415, 1031-1034). Therefore, increased development and differentiation of neuronal stem cells can promote nerve regeneration.

During the developmental stage of the mammalian brain, more than half of developed nerve cells die. In addition, such nerve cell death takes place not only in the nervous system diseases, in particular of aged nervous systems, but also in the normal adult brain (Yuan and Yankner, Nature. 407, 802-809 (2000)). Therefore, apoptosis of nerve cells is a major problem in all nervous system diseases including degenerative brain diseases in the central nervous system and spinal cord and peripheral nervous system injuries. In Europe, transplantation of fetal neuronal stem cells into patients with degenerative brain disease, in particular, Parkinson's disease, has been clinically tried. After transplantation, patients exhibited significant improvement. However, 3 months after transplantation, since most of transplanted cells die, there is a need to continuously transplant neuronal stem cells into patients (Olanow C. W., Kordower J. H., Freeman T. B. (1996) Fetal nigral transplantation as a therapy for Parkinson's disease. Trends Neurosci. 19, 102-109.) In order to survive in the nervous system, transplanted cells must differentiate into their compatible nerve cells to form synapses together with target cells, and participate in electrical signal transmission to continuously receive survival factors from the target cells.

Although many studies on nerve cell apoptosis have been undertaken in differentiated nerve cells, little is known about substances to hinder nerve cell apoptosis, in particular in neuronal stem cells.

Neuronal stem cells divide into other stem cells or cells to be differentiated. At this time, cells suffering from false cell division and unnecessary cells experience cell death. Surviving cells are classified according to types of cells they are differentiated into. Neuronal precursors or neuroblasts, which are differentiated into nerve cells, are differentiated into cells secreting suitable neurotransmitters. Glial precursors, which are differentiated into glial cells, are differentiated into astrocytes and oligodendrocytes. These are cells assisting nerve cells. Astrocytes mechanically and metabolically support nerve cells, and comprise 70-80% of adult brain cells. Oligodendrocytes insulate axons and produce myelin to increase the rate of transmission of signals. Neuronal stem cells in the central nervous systems of fetus and adult can be differentiated into three types of brain cells, depending on environment of brain tissues and type of signals transmitted to neuronal stem cells.

It was reported that there are three types of cells as stem cells in the central nervous system. These cells all exist in the adult rodent brain, and it is believed that the cells exist in the adult human brain. One area containing these cells exists in the brain tissues adjacent to ventricles known as ventricular zones and subventricular zones. Ventricle is spaces through which cerebrospinal fluid can flow. During fetal neurogenesis, rapid cell division takes place in the tissues around the ventricles. In the adult, stem cells around ventricles can exist, but the tissues are very small. The second area in which stem cells exist is not found in humans. The area is rostral migratory stream connecting lateral ventricles and olfactory bulbs in rodents. The third area is the hippocampus, which is associated with memory formation, and exists in both the adult rodent and human brains. Stem cells in the hippocampus exist in the subgranular zones of dentate gyrus. When labeling dividing cells with BrdU (bromodeoxyuridine) in rats, about half of the labeled cells are differentiated into granule cells of dentate gyrus, and 15% are differentiated into glial cells, and the rest do not have particular phenotypes.

Some BrdU-labeled cells in dentate gyrus of human and rat express nerve cell markers such as NeuN, neuron-specific enolase, calbindin, etc. These nerve-like cells are similar to granule cells of dentate gyrus in terms of morphology. The other BrdU-labeled cells express GFAP, which is an astrocyte marker. Recent study has revealed that as a result of analyzing BrdU-labeled cells in the brain tissues of five cancer patients (age 57-72 years) for the purpose of diagnosing, BrdU-labeled cells were most commonly found in the brain of the oldest patient. From this finding, it can be seen that the formation of nerve cells in the hippocampus continues until death. It is known that nerve growth factors are involved in division, differentiation and apoptosis of neuronal stem cells, differentiation of neuronal stem cells into nerve cells and glial cells, and synaptic formation in the development of mammalian nerves.

The receptors for the nerve growth factors are tyrosine kinases. Fibroblast growth factors (FGFs) were first found to be growth factors promoting the division of neuroectoderm and mesoderm-derived cells. FGFs are classified into acidic FGFs (aFGF) and basic FGFs (bFGF) in terms of their isoelectric points. Membrane-associated proteoglycans bind to low-affinity binding sites of FGF receptors, and are essential to FGF's binding with a high-affinity binding site. It is known that almost all high-affinity receptors are receptor-tyrosine kinases, and FGF is bound thereto to form a dimer which causes tyrosine autophosphorylation and transmits signals in 3T3 fibroblast and platelet. FGF receptors express 4 genes into various transcripts by alternative splicing. The receptors can bind with at least one FGF family member, and their ligand binding specificities are determined by their types and splicing forms. FGFs have mitogen activity and induce cell differentiation. The treatment of pheochromocytomas (PC12) with FGF causes their differentiation into cells having neuronal phenotype.

Little is known about the signal transmission system of FGF receptors. When the primary cells of the hippocampus and PC12 cells are treated with FGF receptors, tyrosine phosphorylation increases and p42 MAP kinase (ERK2) and p44 MAP kinase (ERK1), which are mitogen-activated protein kinases (MAP kinase), are activated. Further, it is known that bFGF induces transcription factor such as c-fos. It has been found that FGF increases the survival of the hippocampus and cerebral cortex nerves, and neurite outgrowth in primary nerve cell culture of white rat brain, and decreases excitotoxicity by glutamate. mRNAs of FGF receptors are mainly found in the adult rat brain, in particular in primary cultured nerve cells of developing rat brain and hippocampus. Furthermore, it is known that FGF increases the survival of retinal optic nerves during the development of Xenopus retinal optic nerve cells, and in particular the expression of FGF is drastically increased in a short period of time.

Primary culture of nervous stem cells in E16, in which hippocampal pyramidal nerve cells develop, and treatment of the primary culture with FGF, a nerve growth factor, increase cell division. At this time, 30% of stem cells differentiate into nerve cells, and the remaining stem cells differentiate into glial cells. McKay's group reported that the treatment of with PDGF mainly leads to the differentiation into nerve cells (80%) and the differentiated nerve cells express neuronal markers. They also reported that treatment with FGF and EGF, followed by treatment with CNTF, leads to differentiation into astrocytes, and treatment with thyroid hormone T3 promotes differentiation into oligodendrocytes (Johe, K. K., Hazel, T. G, Muller, T., Dugich-Djordjevic, M. M., McKay, R. D. (1996) Single factors direct the differentiation of stem cells from the fetal and adult central nervous system. Genes Dev 10, 3129-40). These findings mean that PDGF acts as a neurotrophic factor in the early stage of primitive nerve cell development to determine the fate of neuronal cells. The present inventors found that the treatment of the hippocampal primitive nerve cell line (HiB5) with PDGF and FGF inhibits apoptosis of cells and influences the differentiation into nerve cells or glial cells (Kwon, Y. Kim (1997) Expression of brain-derived neutrophic factor mRNA stimulated by basic fibroblast growth factor and platelet-derived growth factor in rat hippocampal cell line, Mol. Cells 7, 320-325.).

Nerve growth factors initiate the division of nerve stem cells, regulate the number of divided cells into apoptosis, initiate the differentiation of divided cells, induce the survival of cells orthodromically moving toward target-derived growth factors and the apoptosis of cells moving in a false direction to regulate the survival of presynaptic nerve cells, and regulate synaptic formation and synaptic remodeling. Since the human central nervous system and peripheral nervous system are hard to regenerate, patients with degenerative brain diseases, and persons crippled due to industrial accidents, traffic accidents and wars, have been social problems. Therefore, special attention has been paid to studies on the regeneration of nervous systems.

Schwann cells play an important role in the generation and regeneration of the peripheral nervous system. During development of embryos, Schwann cells derived from the neural crest previously divide at the sites occupied by axons. That is, axonal growth in the peripheral nervous system depended upon Schwann cells. In particular, Schwann cells produce trophic factors to regulate nerve survival and neurite growth. Axons in nerve cells secrete neuregulin to increase Schwann cell survival and to regulate the ratio between axons and Schwann cells. At this time, Schwann cells receiving no influence from axons die. At the final stage of development, Schwann cells produce myelin sheaths to insulate axons and the differentiation of Schwann cells is completed.

When peripheral nerves are injured in adults suffering from neurogenesis, they undergo Wallerian degeneration at the distal stumps toward nerve endings from the injured sites. However, the proximal stumps toward cell bodies from the injured sites start to regrow. At the distal stumps toward nerve endings from the injured sites, the degenerated axons and myelin sheaths are removes. On the other hand, at the proximal stumps toward cell bodies from the injured sites, the environment is modified to promote axonal regrowth (Kwon, Y et al., “Activation of ErbB2 during Wallerian degeneration of sciatic nerve, J Neurosci, 17:8293-99 (1997); Joung, I. et al., “Effective gene transfer into regenerating sciatic nerves by adenoviral vectors; potentials for gene therapy of peripheral nerve injury,” Mol. Cells, 10:540-45 (2000).

Immediately after nerves are damaged, Schwann cells rapidly divide. Such Schwann cell division is believed to be due to the fact that Schwann cells fail to make contact with axons, or the division is promoted by growth facts secreted from axons. During axonal regrowth, contact of Schwann cells with axons promotes axonal differentiation and regenerates myelin sheaths. Further Schwann cells can influence axonal regeneration from a distance. For example, though nerves are cut and separated by a gap of 1 cm, axons regenerate toward the distal stumps. Such orthodromic movement of axons is possible only when living Schwann cells exist in the distal stump.

Regeneration in the peripheral nervous system occurs in accordance with the following processes: first, Schwann cells are separated from cut axons to obtain division potential (dedifferentiation), axons of nerve cells regrow from injured sites, Schwann cells insulate the regrown axons with myelin sheaths (redifferentiation), and axons grow enough to reach muscles and form neuromuscular junctions at muscle cells.

Chronic and intractable diseases due to increased age of populations cause increased social and economic costs. In particular, therapeutic agents for treating nervous diseases are difficult to develop because of limited knowledge in the neuroscience field.

### SUMMARY OF THE INVENTION

Thus, one object of the present invention to provide a drug and food composition for protecting nerve cells, promoting the differentiation of nerve cells and regenerating nerve cells, comprising berberine, derivatives thereof or pharmaceutically acceptable salts thereof.

It is another object of the present invention to provide a drug and food composition for preventing and treating nervous diseases or nerve injuries, comprising berberine, derivatives thereof or pharmaceutically acceptable salts thereof.

It is yet another object of the present invention to provide a composition for preventing and treating neurodegenerative diseases, ischemic nervous diseases and central or peripheral nerve injuries due to accidents, and for improving learning capability, comprising berberine, derivatives thereof or pharmaceutically acceptable salts thereof.

The composition according to the present invention is useful for preventing and treating physical injuries to the nervous system, including trauma to the head and/or spinal cord, degenerative and ischemic central nerve injuries, peripheral nerve injuries and neuromuscular disorders.

The composition according to the present invention comprises a compound, represented by the following formula 1:

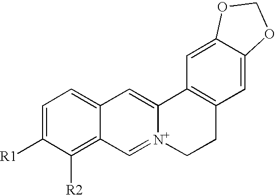

wherein

R1 and R2 are the same or different from each other and independently selected from the group consisting of alkoxy group, alkyl group, hydrogen atom, methylenedioxy group, substituted benzyl group, propoxy group, octyl group, alkenyl group, alkynyl group, amino group, amide group, cyano group, thiocyano group, aldehyde group and halogen atom,

derivatives thereof, or pharmaceutically acceptable salts thereof.

Preferably, the composition according to the present invention comprises berberine wherein R1 and R2 are methoxy group.

The pharmaceutically acceptable salts are preferably acid addition salts formed by suitable pharmaceutically acceptable free acids. The compound of formula 1 can be formed into pharmaceutically acceptable acid addition salts in accordance with known processes in the field. The pharmaceutically acceptable free acids may be inorganic or organic acids, and in particular include hydrochloric acid, nitric acid, hydrobromic acid, sulfuric acid, phosphoric acid, citric acid, acetic acid, lactic acid, tartaric acid, maleic acid, fumaric acid, formic acid, propionic acid, oxalic acid, trifluoroacetic acid, benzoic acid, gluconic acid, methanesulfonic acid, glycolic acid, succinic acid, 4-toluenesulfonic acid, galacturonic acid, embonic acid, glutamic acid, aspartic acid, etc.

Examples of pharmaceutically acceptable salts are preferably berberine sulfate and berberine chloride, and more preferably berberine chloride.

Berberine (7,8,13,13a-tetradihydro-9,10-dimethoxy-2,3-(methylenedioxy) berbinium) is an alkaloid extracted from plants such as Berberidaceae, Coptidis Rhizoma, Hydrastis canadensis L., Phellodendron amurense, etc. Berberine has been traditionally used as a dye in wool, silk and leather industries, and developed as an antibiotic, an antipyretic, an antidiabetic and an anticancer drug in medicine industry.

The present inventors identified protective and differentiative effects of berberine on neuronal stem cells and nerve cell lines cultured in vitro in stress models (serum deprivation-induced stress and oxidative stress). In in vivo experiments, the present inventors identified the neuroprotective effect of berberine in animal models for dementia and stroke. Further, In in vitro experiments, the present inventors examined the inhibitory effects of berberine against apoptosis induced by serum deprivation-induced stress and oxidative stress, and the effect of berberine on differentiation and regeneration of nerve cells and protective an effect against neurotoxins, by treating human neuroblastomas (SH-SY5Y), white rat neuronal stem cells (HiB5), and PC12 cells with berberine. Furthermore, the present inventors identified the recovery and protective effects of berberine against apoptosis of nerve cells due to Alzheimer's disease and ischemic stroke, and effects of berberine on survival and differentiation of neuronal stem cells and nerve cells.

From these experiments, it is believed that berberine, derivatives thereof or pharmaceutically acceptable salts thereof will be useful for preventing and treating nervous diseases and injuries including nervous system disorders, degenerative brain diseases, trauma to the head an/or spinal cord, neuromuscular disorders, etc.

### BRIEF DESCRIPTION OF THE DRAWINGS

The above and other objects, features and other advantages of the present invention will be more clearly understood from the following detailed description taken in conjunction with the accompanying drawings in which:

FIG. 1 is a graph quantitatively showing the extent to which berberine inhibits apoptosis of nerve cells induced by MK-801 (0.5 mg/kg) in the cerebral slice of white young rat;

FIG. 2 is a result of RT-PCR showing the expression of bcl-2 mRNA, an anti-apoptosis gene expressed in cerebral tissues of white rats, after intraperitoneally injecting berberine (upper panel). This figure reveals that the expression of bcl-2 mRNA is higher than in the control group. The lower panel shows the expression of GAPDH mRNA.

FIG. 3 is a graph showing the effect of berberine against apoptosis in white rat-derived neuronal stem cells (HiB5);

FIGS. 4a and 4b are graphs showing the protective effect of berberine against apoptosis induced by stresses (FIG. 4a: serum deprivation-induced stress, and FIG. 4b: oxidative stress) in human neuroblastoma SH-SY5Y, which is a differentiated nerve cell line; For FIG. 4a, 1: Serum; 2: N2; 3: N2+retinoic acid 5 μM; 4: N2+berberine 0.25 μg/ml; 5: N2+berberine 1.5 μg/ml; 6:N2+berberine 3 μg/ml. For FIG. 4b, 1: N2; 2: N2, H₂O₂ μM; 3: N2, H₂O₂+Retinoic acid 5 μM; 4: N2, H₂O₂+Berberine 0.25 μg/ml; 5: N2, H₂O₂+Berberine 1.5 μg/ml; 6: N2, H₂O₂+Berberine 3 μg/ml; 7: N2, H₂O₂+Berberine 3.5 μg/ml;

FIGS. 5a and 5b are graphs showing the protective effect of berberine against apoptosis induced by stresses (FIG. 5a: serum deprivation-induced stress, and FIG. 5b: dexamethasone stress, a derivative of stress hormone (glucocorticoid) in pheochromocytomas (PC12) derived from white rat neural crest; for FIG. 5a, 1: serum; 2: RPMI; 3: RPMI+NGF 100 ng/ml; 4: RPMI+Berberine 0.25 μg/ml; 5: RPMI+Berberine 1.5 μg/ml; 6: RPMI+Berberine 3 μg/ml. For FIG. 5b, 1: N2; 2: N2+Dexamethasone 1 μM; 3: N2+Dexamethasone+NGF 100 ng/ml; 4: N2+Dexamethasone+Berberine 0.25 μg/ml; 5:: N2+Dexamethasone+Berberine 1.5 μg/ml; 6: N2+Dexamethasone+Berberine 3.5 μg/ml.

FIG. 6 is confocal microscopic images showing the effect of berberine on inducing differentiation of HiB5 nerve cells. bFGF+ represents bFGF (basic fibroblast growth factor)—treated cells, and bFGF− represents bFGF-untreated cells;

FIG. 7 is a graph showing the effect of berberine on inducing differentiation of HiB5 nerve cells;

FIG. 8 is confocal microscopic images showing the effect of berberine on neurite regeneration in human neuroblastoma SH-SY5Y, which is a differentiated nerve cell line. Retinoic acid is a positive control group which causes the neurite differentiation of SH-SY5Y;

FIG. 9 is a graph showing the effect of berberine on neurite regeneration in human neuroblastoma SH-SY5Y, which is a differentiated nerve cell line;

FIG. 10 is microscopic images showing the neuroregenerative effect of berberine, after staining the brain tissues of dementia-induced white rats with hematocylin. In FIG. 12, regions used for cell count after fluorescence-staining the brain tissues of dementia-induced white rats were represented as squares;

FIG. 11 is confocal microscopic images showing the neuroregenerative effect of berberine, after fluorescence-staining the brain tissues of dementia-induced white rats with calbindin antibody, which is a nerve-specific marker;

FIG. 12 is a graph showing the neuroregenerative effect of berberine in the brain tissues of dementia-induced white rats; Ibo=Ibotenic acid; B=Berberine; DG=Dentate Gyrus; ENT=Entorhinal cortex;

FIG. 13 is confocal microscopic images (×200) showing the neuroregenerative effect of berberine, 1 week after intraperitoneally injecting berberine into sciatic nerve-damaged white rats. White lines indicate axons longer than 300 μm stained with beta-tubulin isotypeIII (red), and arrowheads indicate degenerated myelin sheaths stained with MBP (myelin binding protein, green) antibody;

FIG. 14 is confocal microscopic images (×200) showing the neuroregenerative effect of berberine, 2 week after intraperitoneally injecting berberine into sciatic nerve-damaged white rats. White lines indicate axons longer than 300 μm stained with beta-tubulin isotypeIII (red), and arrows indicate regenerated myelin sheaths of Schwann cells longer than 200 μm stained with MBP (myelin binding protein, green) antibody;

FIG. 15 is confocal microscopic images (×200) showing the neuroregenerative effect of berberine, 4 weeks after intraperitoneally injecting berberine into sciatic nerve-damaged white rats. White lines indicate axons longer than 300 μm stained with beta-tubulin isotypeIII (red), and arrowheads indicate myelin sheaths longer than 200 μm stained with MBP (myelin binding protein, green) antibody. It was confirmed that the number of long and thick axons and myelin sheaths increased. When myelin sheaths were differentiated and then insulated regrowing axons, two antibody markers were overlapped to appear to be yellowish;

FIG. 16 is magnified (×400) views of FIG. 13;

FIG. 17 is magnified (×400) views of FIG. 14;

FIG. 18 is magnified (×400) views of FIG. 15;

FIGS. 19a and 19b are graphs showing the neuroregenerative effect of berberine, 1, 2 and 4 weeks, respectively, after intraperitoneally injecting berberine into sciatic nerve-damaged white rats. FIG. 19a represents the number of axons longer than 300 μm. FIG. 19b represents the number of myelin sheaths longer than 200 μm;

FIG. 20 is photographs showing the neuroregenerative effect of berberine during reformation of neuromuscular junctions. In the control group, nerve endings reached only one muscle fiber, but did not spread to other fibers. In the group administered with berberine, the nerve endings reached all muscle fibers to form neuromuscular junctions; and

FIG. 21 is microscopic images showing the protective effect of berberine against apoptosis in animal models for brain ischemia. A and B represent CA1 in the normal hippocampus, C and D represent CA1 in the ischemia-induced hippocampus, and E and F CA1 in the hippocampus intraperitoneally injected with berberine after ischemia-induced. In this figure, sites for magnitude were indicated as open squares; and

FIG. 22 is a graph showing the effect of berberine against apoptosis in animal models for brain ischemia.

### DETAILED DESCRIPTION OF THE INVENTION

Hereinafter, the present invention will be explained in more detail.

### 1. Regenerative and Protective Effects of Berberine Against Apoptosis of Brain Nerve in MK-801 Model

In a young white rat brain administered with berberine alone, apoptosis of nerve cells was not observed through TUNEL staining, unlike nerve cells of a young white rat brain damaged by MK-801. It was observed that berberine considerably inhibits apoptosis of nerve cells induced by MK-801.

Further, it was observed that bcl-2 mRNA, an anti-apoptosis gene, was increased in cerebral tissues by administration of berberine. The mechanisms by which nerve cell growth factors inhibit apoptosis of nerve cells are as follows: 1) inhibition of death effector gene expression, and 2) promotion of cell survival promoting genes (e.g., bcl-2, bcl-xL, etc) expression. Therefore, it is assumed that berberine functions as a nerve growth factor, and berberine increases the production of Bcl-2, a representative anti-apoptosis protein, thereby efficiently inhibiting apoptosis of nerve cells.

### 2. Neuroregenerative Effect by Survival and Differentiation of Neuronal Stem Cells

In order to evaluate the effect of berberine on differentiation and regeneration of nerve cells, neuronal stem cell line (HiB5) derived from white rat hippocampus was used. In order to examine the effect of berberine against apoptosis and the protective effect of increasing cell survival, HiB5 cells were treated with berberine during culturing under conditions for initiation of differentiation. The protective effect of berberine against apoptosis of HiB5 cells was measured by MTT (3-(4,5-dimethylthiazol-2-yl)-2,5-diphenyl tetrazolium bromide) assay.

A control group was treated with bFGF to increase cell survival and to induce the differentiation into nerve cells. As a result, the survival rate of HiB5 cells in the control group increased 1.6 times, whereas the group treated with berberine exhibited the protective effect against apoptosis of nerve cells (about two times). Therefore, it is believed that berberine has a protective effect against apoptosis during differentiation of neuronal stem cells and an effect of increasing cell survival.

### 3. Protective Effect of Berberine Against Apoptosis Induced by Stress in Differentiated Nerve Cell Line

### 1) Survival of Human Neuroblastomas Under Condition of Serum Deprivation-Induced Stress

In order to prepare a stroke-like cell model using brain-derived human neuroblastomas (SH-SY5Y), the cells were cultured under serum deprivation. After the culture was treated with berberine, cell survival was evaluated by performing the MTT assay to identify the protective effect against cell injuries. Retinoic acid, which increases cell survival as a cell differentiation factor, was used as a positive control for differentiation. 3 hours before depriving serum, the cells were treated with berberine, and cultured in chemically defined media, N2, for 2 days to induce nerve regeneration.

As a result, the cell survival rate in the group treated with berberine was two times higher than in the control group.

Therefore, it can be seen that berberine has an excellent protective effect against apoptosis, a positive effect on cell survival and a neuroprotective effect.

In addition, after rat PC12 cells were treated with berberine under condition of serum deprivation-induced stress, the protective effect of berberine against cell injuries was examined. NGF, increasing cell survival and inducing cell differentiation, was used as a positive control group. As a result, the cell survival rate in the group treated with berberine was 1.5 times higher than in the control group.

Therefore, it is believed that berberine has an excellent neuroprotective effect in PC12 cells.

### 2) Survival of SH-SY Cells Under Condition of Oxidative Stress

Apoptosis of nerve cells is a cause of degenerative brain diseases. A common cause of apoptosis is oxidative stress. Since H₂O₂ is a strong oxidant and produces oxidative stress in cells, it is used to prepare a cell model for degenerative brain diseases such as stroke and dementia. In the present invention, SH-SY5Y cells were treated with H₂O₂ to induce cell injuries. Three hours before the treatment, berberine was added to cell culture solution to examine its protective effect against oxidative stress.

As a result, the cell protection effect against apoptosis in the group treated with berberine was more than three times higher than in the control group. Therefore, it can be seen that berberine has an excellent protective effect against apoptosis of neuroblastomas and a positive effect on cell survival under condition of oxidative stress.

### 3) Effect of Berberine on Survival of Neuroblastoma Cells Under Condition of Glucocorticoid Stress

Glucocorticoids are hormones secreted from the adrenal gland, and involve in a variety of metabolic processes including glycometabolism. In particular, stress causes release of glucocorticoids to reduce cell divisions of neuronal stem cells and induce apoptosis of nerve cells in the brain. In the present invention, PC12 cells were treated with dexamethasone, a glucocorticoid derivative, to examine the effect of berberine against apoptosis of nerve cells. 3 hours after treatment with berberine, dexamethasone was added to a culture solution and incubated for 2 days to induce neuroregeneration.

The cell survival rate in the group treated with NGF as a positive control increased about 1.5 times, whereas the cell survival rate in the group treated with berberine increased about 1.3 times. Therefore, it is believed that berberine can increase survival of rat PC12 cells under condition of dexamethasone stress.

### 4. Regenerative Effect of Berberine During Differentiation of Nerve Cells

The effect of berberine on differentiation of nerve cells and the effect of berberine on the regeneration of neurites were evaluated using neuroblastomas.

### 1) Induction of Differentiation

In order to evaluate the effect of berberine on inducing differentiation of neuronal stem cells, HiB5 cells were cultured under conditions for initiation of differentiation for 1 day. After the culture was treated with berberine and further cultured for 2 days, the number of cells having neurites two times longer than their cell bodies was counted. A positive control group was treated with bFGF to induce the differentiation into nerve cells. As a result, cell bodies got smaller and neurites got longer in the group treated with bFGF, whereas the number of cells differentiated into nerve cells increased two times in the group treated with berberine. Therefore, it can be seen that berberine has an excellent effect of promoting differentiation of neuronal stem cells into nerve cells.

### 2) Effect on Neurite Regeneration

In order to evaluate the effect of berberine on neurite regeneration, SH-SY5Y cells were used in accordance with the same manner as described above. Retinoic acid inducing neurite growth were used as a positive control group. It was observed that total number of nerve cells doubled and the number of cells having neurites three times longer than their cell bodies has increased about 1.7 times, in the group treated with berberine.

### 5. Neuroregenerative Effect of Berberine in Animal Model for Dementia

A common characteristic of Alzheimer's disease in the early stages is memory loss. As a part of the limbic system responsible for learning and memory, the hippocampus is involved in the formation of short-term and long-term memories. Degeneration in the hippocampus and forebrain are most commonly found in the brain of Alzheimer's patients, and senile plaques are most commonly found in the hippocampus and forebrain. In particular, degeneration of nerve cells in the CA1 and entorhinal cortex of the hippocampus is fastest. Since survival of cholinergic neurons projecting from basal forebrain depends on NGF and BDNF, which are target-derived neurotrophic factors, cholinergic neurons are rapidly degenerated in patients suffering from Alzheimer's disease.

Many etiological studies on initiating factors of Alzheimer's disease have been carried out. Among them, many experiments have noted that abnormal phosphorylation of β-amyloid, which is a main component of senile plaques, or tau proteins found in the neurofibrillary tangles of dying nerve cells, is associated with apoE4, etc. However, there exist too many genes related with Alzheimer's disease, and no initiating factors and gene mutations commonly found in all patients have been found. Currently used therapeutic agents of Alzheimer's disease include acetylcholine esterase inhibitors for enhancing activities in the cholinergic signal transmission system, acetylcholine esterase precursors, and a drug for improving energy metabolism of nerve cells. However, these therapeutic agents may only transiently alleviate symptoms. Therefore, there is a need for neurotrophins or nerve cell stimulants capable of increasing nerve cell survival in order to reduce Alzheimer's disease progress and etiologically treat Alzheimer's disease. Among them, nerve growth factors have drawn attention. So far, clinic trials with NGF have shown some effects in cholinergic neurons (Knusel, B. and Gao, H. (1996) Neurotrophins and Alzheimer's disease: beyond the cholinergic neurons. Life Sci. 58, 2019-2027; Lapchak, P. A. (1993) Nerve growth factor phamacology: application to the treatment of cholinergic neurodegeneration in the Alzheimer's disease. Exp Neurol 124, 16-20), but did not exhibit satisfactory effects (Neve et al., (1996) A comprehensive study of the spatiotemporal pattern of beta-amyloid precursor protein mRNA and protein in the rat brain: lack of modulation by exogenously applied nerve growth factor. Brain Res Mol Res. 39, 185-197). Therefore, it is necessary to select substances capable of protecting against apoptosis of nerve cells, increasing survival and regeneration of nerve cells, and increasing the survival and differentiation of neuronal stem cells.

In order to reduce neurodegeneration by Alzheimer's disease and promote neuroregeneration in the hippocampus responsible for learning and memory, the present inventors identified the protective effect of berberine against apoptosis, and the neuroregenerative effect of berberine in an animal model for dementia.

In order to prepare the animal model for dementia, ibotenic acid, a kanate derivative, was microinjected into the entorhinal cortex of adult rat brain using a stereotaxic frame. As a result, it was seen that 2 weeks after microinjection, calbindin-positive neurons were reduced by 30˜40% in the hippocampus and entorhinal cortex, and 4 weeks after microinjection, some of them were recovered. Cell numbers in the dentate gyrus regions slowly reduced, compared with cell numbers in the CA1 region. However, in the case of injecting berberine, survival rates of pyramidal cells in the CA1 region increased 2.5 times, and those of granule cells in the dentate gyrus regions increased two times. Since increase of survival rates of dying nerve cells leads to cell regeneration, it is believed that berberine has a protective effect against apoptosis of nerve cells and a neuroregenerative effect in an animal model for dementia.

### 6. Effect of Berberine on Regeneration of Sciatic Nerves in the Peripheral Nervous System

Since the central nervous system and peripheral nervous system are hard to regenerate, degenerative brain diseases, and persons crippled due to industrial accidents, traffic accidents and wars, have been social problems. Therefore, special attention has been paid to studies on the regeneration of nervous systems.

The present inventors examined whether berberine promotes axonal regrowth, the regeneration of myelin sheaths, and the formation of neuromuscular junctions in muscle cells, in the regeneration process of sciatic nerves through which most nerve fibers pass in the peripheral nervous system.

The present inventors observed the degree of nerve regeneration 1 week, 2 weeks and 4 weeks after intraperitoneally injecting PBS (phosphate-buffered saline) or berberine into sciatic nerves of a rat. As a result, it was observed that 4 weeks after injecting, the number of neurites longer than 300 μm had doubled, and that of myelin sheaths longer than 200 μm had doubled (1 week) and increased 3 times (4 weeks). Therefore, it can be seen that berberine promotes axonal growth and the regeneration of myelin sheaths during peripheral nerve regeneration.

In order to see if berberine influences the regeneration of nerve endings at neuromuscular junctions, 4 weeks after operation, the present inventors separated hind limb muscle connected to sciatic nerve. As a result, it was observed in the control group that nerve endings were stained, but did not spread to muscle fibers and thus did not form neuromuscular junctions. In the group administered with berberine, the nerve endings spread to all muscle fibers.

Therefore, it is believed that berberine promotes axonal growth, the regeneration of myelin sheaths and the regeneration of nerve endings to form neuromuscular junctions during regeneration of peripheral nerves.

### 7. Neuroregenerative Effect of Berberine in Animal Model for Stroke

In the present invention, white rats suffering from forebrain ischemia induced by 4-vessel occlusion (4-VO) were used to examine cell injuries (Pulsinelli, W. A. and Buchan, A. M. (1988) The four-vessel occlusion rat model: method for complete occlusion of vertebral arteries and control of collateral circulation. 19, 913-4).

CA1 pyramidal neurons of the hippocampus are most susceptible to ischemia, and undergo cell death 72 hours after reperfusion. In order to observe delayed neuronal death in the hippocampal CA1 region, 1 week after reperfusion, the time when almost all nerve cells were damaged, the rats were sacrificed and tissue sections from the hippocampus were observed under an optical microscope. Normal nerve cells were observed in sham operated white rats having undergone no ischemia. However, in the hippocampal CA1 region of rats treated with physiological saline after ischemia induction, apoptosis of nerve cells was observed. This observation can be seen through morphological changes in nerve cells.

On the contrary, in the hippocampus of rats treated with berberine, the nerve cells were similar to normal nerve cells in terms of their morphology. These results show that berberine has a protective effect against nerve cell injuries in the hippocampal CA1 region induced by 4-VO.

### 8. Role of Nerve Growth Factors and Berberine in the Nerve Regeneration

Nerve growth factors initiate the division of neuronal stem cells, regulate the divided cells into apoptosis, induce the survival of cells orthodromically moving toward target-derived growth factors and apoptosis of cells moving in a false direction to regulate the survival of presynaptic nerve cells, and regulate new synaptic formation and remodeling. Since berberine induces the differentiation of neuronal stem cells, inhibits apoptosis and promotes neurite differentiation, it is expected that berberine will perform functions of nerve growth factors.

On the other hand, as a result of examining the toxicity and side effects of berberine through in vivo experiments using white rats, it was observed that berberine had no acute toxicity and no side effects on liver functions. When intraperitoneally injected, LD₅₀ value of berberine was 24.3 mg/kg and proved to be safe. The dosage for berberine can be varied depending upon known factors, such as age, sex, body weight, disease severity and health condition of the recipient. The daily dosage is commonly in the range of 100 to 150 mg/60 kg of body weight in two or three installments.

Berberine, derivatives thereof or pharmaceutically acceptable salts thereof may be mixed with an appropriate carrier or excipient, or may be diluted in an appropriate diluent. Examples of the carrier, excipient and diluent include lactose, dextrose, sucrose, sorbitol, mannitol, xylitol, erythritol, maltitol, starch, acacia gum, alginate, gelatin, calcium phosphate, calcium silicate, cellulose, methyl cellulose, amorphous cellulose, polyvinyl pyrrolidone, water, methylhydroxy benzoate, propylhydroxy benzoate, talc, magnesium stearate and mineral oils. The composition according to the present invention can further comprise fillers, anti-coagulating agents, lubricants, wetting agents, flavors, emulsifying agents, preservatives, etc. For fast or sustained release of active ingredients into a mammal, the composition according to the present invention can be formulated in accordance with well-known processes.

The formulation may be in dosage form such as tablets, powders, pills, sachets, elixirs, suspensions, emulsions, solutions, syrups, aerosols, soft or hard gelatin capsules, sterile water for injection, sterilized powders, etc. The composition according to the present invention may be administered through a suitable route such as oral, transdermal, subcutaneous, intravenous or intramuscular route. In the present invention, berberine, derivatives thereof or pharmaceutically acceptable salts thereof may be formulated into pharmaceutical preparations for preventing and treating nervous system diseases, or may be added to foods or beverages. The composition according to the present invention may be used as drugs or foods to treat degenerative brain diseases such as dementia, chronic epilepsy, palsy, ischemic brain diseases, Parkinson's disease and Alzheimer's disease. Examples of foods include beverages, gums, teas, vitamin complexes, health care products, etc.

The present invention is illustrated in greater detail below with reference to Examples. These Examples are provided only for illustrative purposes, but are not to be construed as limiting the scope of the present invention.

### EXAMPLE 1

### General Methods

### 1) Berberine Chloride (Product #: B3251, Sigma-Aldrich, U.S.A) was Used

### 2) Nerve Cell Line Culture

Since HiB5 cells derived from white rat embryonic hippocampus were prepared by retroviral infection of temperature sensitive SV40 large T antigen, they divided at 34° C., but the cell division stopped at 39° C. (body temperature of rat). When bFGF (20 ng/ml) was added to the HiB5 cells, cell survival increased and HiB5 cells differentiated into nerve cells to express marker molecules of nerve cells. Cell culture medium was prepared by adding a mixture of 10% FBS (fetal bovine serum), penicillin/streptomycin, glutamine and sodium pyruvate (0.11 g/L) to DMEM. On differentiating at 39° C., another cell culture medium was prepared by adding pyruvate to a serum-free medium (N2, containing DMEM/F12, insulin, transferrin, Putreseine and BSA; Botten Stein & Sato., 1979). PC12 cells and SH-SY5Y cells were cultured in DMEM supplemented with 10% FBS. In order to differentiate the cells, NGF or retinoic acid was added to a serum-free medium.

### 3) MTT Assay of Nerve Cell Line

Cell injuries under condition of serum deprivation-induced stress and oxidative stress were measured by MTT assay. For MTT assay, Cells were seeded into N2 medium in an amount of 7.5×10³ cells, and then a neurotoxin or a nerve growth factor was added thereto to differentiate the cells and then 0.1 volume of 4 mg/ml MTT was further added. The mixture was stored at 37° C. for 3-4 hours. After 100 μl of solubilization buffer was added to the mixture and then stored for 24 hours, O.D. values were measured by ELISA assay.

### 4) Preparation of Animal Model for Dementia

After white rat brain was fixed with a stereotaxic frame, 1.5 μl (1 mg/ml) of ibotenic acid and PBS, or 1.5 μl (1 mg/ml) of berberine was injected into the entorhinal cortex region using a microinjector to prepare a model for dementia. 2 weeks after operation, the animal was perfused.

### 5) Immunostaining

Brain was fixed with 4% paraformaldehyde and cryosected to a thickness of 40 μm, and then the cryosected brain was permeablized with 0.5% Triton X-100 for 20 minutes and blocked using 15% blocking serum for 2 hours. After reacting with anti-calbindin antibody at a temperature 4° C. for 16 hours and then further reacting with FITC-labeled secondary antibody or rhodamin (TRITC)-labeled secondary antibody for 1 hour, the cells were fixed on a slide before examining under a confocal microscope.

### 6) Sciatic Nerve Crush in White Rat

After a Sprague-Dawley white rat (male, weighing about 200 g) was anesthetized with pentobarbital (50 mg/kg), the left sciatic nerve was exposed at the sciatic notch. Subsequently, all nerve fibers except the artery in the sciatic nerve were cut, or both sides of nerve fibers were tied using #9 blood vessel suture, and then the center of the nerve fibers were cut using iridectomy scissors. In a crush model, nerve fibers were thoroughly crushed twice using a crush clip. After nerve fibers were injured, proximal stumps and distal stumps were obtained over various time intervals (6 hours, 1 day, 3 day, 7 day, 14 day, 21 day and 28 day), respectively, before testing. For comparison, the right sciatic nerve was used as a control group.

### EXAMPLE 2

### Regenerative and Protective Effects of Berberine on Cranial Nerve Cells Using MK-801 Model

### 1) Model for Apoptosis Induced by MK-801

MK-801 reaches maximal concentrations in plasma and brain within 10 to 30 minutes of injection with an elimination half-life of 1.9 hours (Vezzani, A., Serafini, R., Stasi, M. A., Caccia, S., Conti, I., Tridico, R. V. and Samanin, R. (1989) Kinetics of MK-801 and its effect on quinolinic acid-induced seizures and neurotoxicity in rats. J Pharmacol Exp Ther 249, 278-83). Ikonomidou et al. found that when MK-801 was administered to a young rat (7-8-days old) to inhibit NMDA receptors (for 2-3 hours), nerve cells highly sensitive to NMDA receptors died through apoptotic neurodegeneration. At this time, the number of dead nerve cells amounted to 12-26% of total nerve cells (Ikonomidou, C., Bosch, F., Miksa, M., Bittigau, P., Vockler, J., Dikranian, K., Tenkova, T. I., Stefovska, V., Turski, L. and Olney, J. W. (1999) Blockade of NMDA receptors and apoptotic neurodegeneration in the developing brain. Science 283, 70-4).

### 2) The Protective Effect of Berberine on Nerve Cells was Evaluated Using Models for Apoptosis of Nerve Cells Induced by MK-801 in 7-Day Old White Rats

Young rats were divided into 5 groups: a) a group administered with physiological saline alone, b) a group administered with MK-801 (0.5 mg/kg) alone, c) a group administered with berberine (5 mg/kg) alone, and d) a group pretreated with berberine (5 mg/kg) and then administered with MK-801 (0.5 mg/kg). All groups were intraperitoneally injected.

Experimental animals were sacrificed under anesthetization and their brains were excised. The excised brains were fixed with formalin and tissue sections were obtained. The tissue sections were stained by TUNEL method, and photographed (×1.25 and ×400) using an optical microscope (Olympus BX 50).

The results are shown as follows:

i) The group administered with physiological saline alone had normal cerebral sections as shown by TUNEL staining in cerebral sections of 7-day old white rats.

ii) The group administered with MK-801 alone:

1 week after intraperitoneally injecting MK-801 (0.5 mg/kg) into 7-day old white rats, apoptosis of nerve cells in cerebral slices was identified.

Black cells, representing cells positive to the TUNEL method which stains only cells having segmented DNA in nuclei,were seen.

iii) The group administered with berberine alone

5 days after intraperitoneally injecting berberine (5 mg/kg) into 7-day old white rats, the cerebral slices were stained with the TUNEL method. Berberine did not induce apoptosis of nerve cells.

iv) The group pretreated with berberine and then administered with MK-801

After 3-day old white rats were pretreated with berberine (5 mg/kg) for 5 days and intraperitoneally administered with MK-801 (0.5 mg/kg) to the rat, the cerebral slices were observed. As a result, it was seen that berberine inhibits apoptosis of nerve cells induced by MK-801 .

### EXAMPLE 3

### Quantitative Comparison of Nerve Cell Apoptosis in White Rat Cerebra

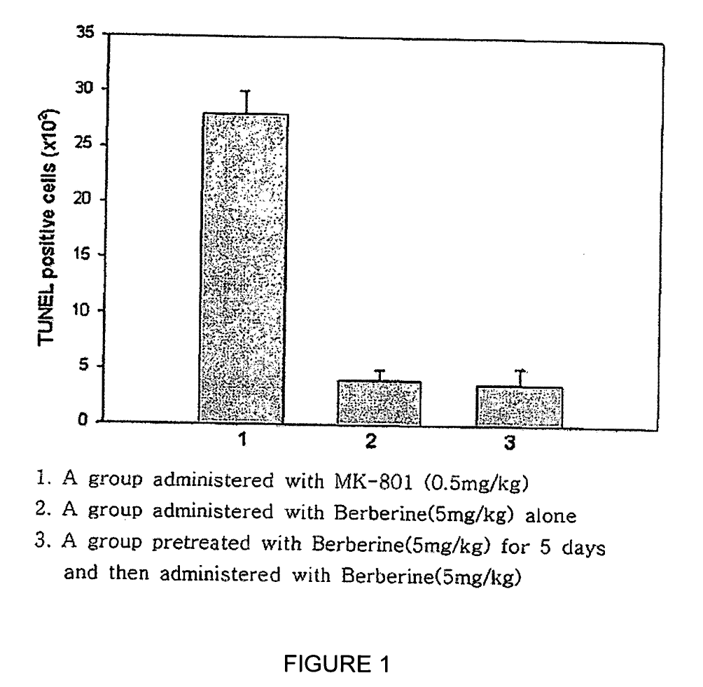

A group administered with MK-801 (0.5 mg/kg), a group administered with berberine (5 mg/kg) for 5 days, and a group pretreated with berberine (5 mg/kg) for 5 days and then administered with MK-801 (0.5 mg/kg), were used to quantitatively compare the inhibition of nerve cell apoptosis by berberine. The number of TUNEL-positive dead nerve cells in the same area of cerebral slices of 12 rats per group was counted, and the numbers were averaged (FIG. 1).

### EXAMPLE 4

### Expression of bcl-2 mRNA and GAPDH mRNA in White Rat Cerebra

7-day old rats were divided into 4 groups: a) a group administered with physiological saline alone, b) a group administered with MK-801 (0.5 mg/kg) alone, c) a group administered with berberine (5 mg/kg) alone for 2 and 5 days, respectively, and d) a group pretreated with berberine (5 mg/kg) for 5 days and then administered with MK-801 (0.5 mg/kg). All groups were intraperitoneally injected. RT-PCR was performed to examine the expression of bcl-2 mRNA, which is an anti-apoptosis gene expressed in cerebral tissues. GAPDH mRNA was used as a control group. The expression of GAPDH mRNA was also performed by RT-PCR method.

RT-PCR

### i) Total RNA Isolation

1 ml of TRI Reagent (Molecular Research Center Inc., USA) was added to 100 mg of cerebral tissue sections, and the mixture was homogenized and then left at room temperature for 10 minutes. 0.1 ml of BCP (Sigma, USA) was added to 1 ml of the homogenized mixture, mixed with each other for 1 minute, and then left at 4° C. for 10 minutes. After the mixture was centrifuged at 12,000 rpm, 4° C. for 15 minutes, the supernatant was added to cold isopropanol and left at a temperature of −20° C. for 16 hours. Thereafter, the supernatant was centrifuged at 12,000 rpm, 4 for 15 minutes to obtain RNA precipitates. The obtained RNA precipitates were washed with DEPC diethylpyrocarbonate)-treated cold ethanol (75%), and dried using SpeedVac. The dried RNA was dissolved in DEPC-treated distilled water. After the concentration and purity of RNA were spectrophotometrically measured at 260 nm, the isolated RNA was stored at a temperature of −20° C. before use.

### ii) cDNA Synthesis (Reverse Transcription: RT)

2 μg of total RNA obtained above was mixed with 4.0 μl of 5× RT buffer, 1.0 μl of oligo (dT16) (100 pmoles/μl), 4 μl of 10 mM dNTPs (Promega, USA), 0.5 μl of RNasin (40 Units/μl, Promega, USA) and 1.0 μl of MMLV reverse transcriptase (200 units/μl, Promega, USA), and DEPC-treated distilled water was added thereto until a total volume of the reaction solution was 30 μl. The reaction was performed in a DNA thermal cycler (Perkin Elmer 2400, USA) at 42° C. for 1 hour to synthesize cDNA.

### iii) Polymerase Chain Reaction: PCR

1 μl of RT product was mixed with sense and antisense primers (each 10 pmoles), 1 μl of 10 mM dNTPs, 2 μl of 10× buffer (20 mM Tris-Cl, 1.5 mM MgCl₂, 25 mM KCl, 0.1 mg/ml gelatin, pH 8.4) and 1 unit of Taq DNA polymerase (Promege, USA), and then distilled water was added thereto until a total volume of the reaction solution was 25 μl. Polymerase chain reaction was performed using a DNA thermal cycler (Perkin Elmer 2400, USA).

### iv) Electrophoresis and Analysis

10 μl of amplified PCR product was electrophoresed in a 1.5% agarose gel, and the density was measured using a gel documentation system (Bio-Rad Lab, USA).

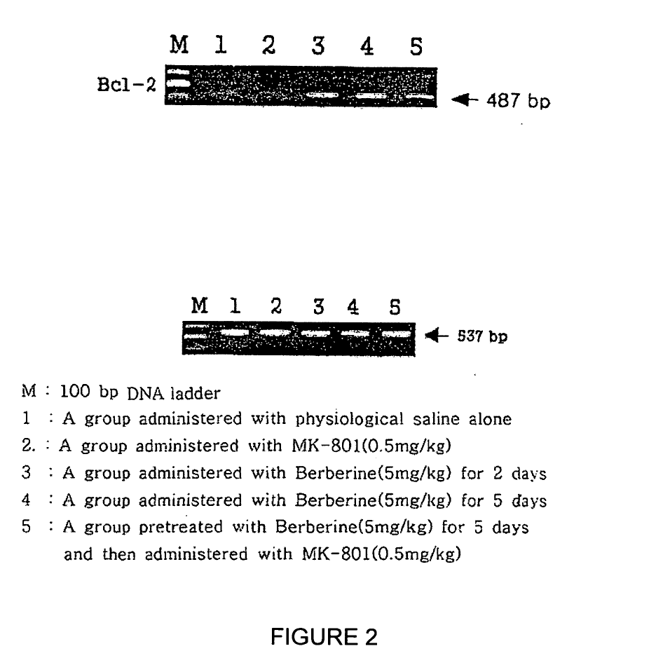

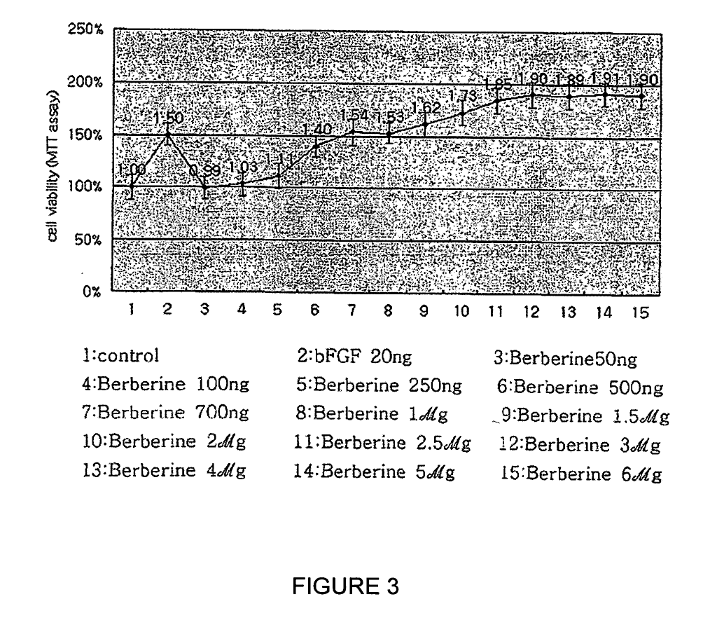

### 2) As a Result, the Group Treated with Berberine Shows High bcl-2 mRNA Expression, Compared with the Group Treated with MK-801 (FIGS. 2a and 2b)

### EXAMPLE 5

### Regenerative Effect of berberine by Survival and Differentiation of Neuronal Stem Cells

HiB5 cell line used in this experiment was prepared by infecting primary cultured cells of temperature sensitive SV40 large T antigen in rat embryonic hippocampus (embryonic day 16) using retroviral vectors. The HiB5 cell line was divided at the permissive temperature (32° C.), but the cell division stopped at the non-permissive temperature (body temperature of rat: 39° C.). A small number of GABAegic neurons differentiated in the rat embryonic hippocampus (embryonic day 16), and glutamatergic pyramidal cell precursors still divided, some of which penetrated into dentate gyrus regions through dentate migration pathways in embryonic day 18 to differentiate into glutamatergic granule cells (Altman J., Bayer. S. A. (1990a) Prolonged sojourn of developing pyramidal cells in the intermediate zone of the hippocampus and their settling in the stratum pyramidale. J Comp Neurol. 301, 343-64.; Altman J., Das G. D. (1965) Post-natal origin of microneurones in the rat brain, Nature. 28, 953-956.). When cells stop their cell-division and start to differentiate in vivo, apoptosis occurs. In HiB5 cell line, the number of cells undergone cell death in 2 days after the differentiation amounts to 60-70%.

In order to evaluate the protective effect of berberine against apoptosis and the effect of increasing cell survival, HiB5 cells were cultured under conditions for initiation of differentiation. At this time, the culture was treated with berberine at various concentrations (50 ng/ml˜6 μg/ml). The protective effect of berberine against apoptosis was measured by MTT assay. A positive control group was treated with bFGF to block apoptosis, increase cell survival and induce the differentiation into nerve cells.

Since HiB5 neuronal stem cells undergo cell death during the differentiation, the cells were considered as a negative control group. The percentage of O.D. values is determined for the groups treated with bFGF and berberine, respectively.

TABLE 1

| Sample | Control | bFGF | Berbeline | | | | | | | | | | | | |
|---|---|---|---|---|---|---|---|---|---|---|---|---|---|---|---|
| Concen- | | 20 ng | 50 ng | 100 ng | 250 ng | 500 ng | 700 ng | 1 μg | 1.5 μg | 2 μg | 2.5 μg | 3 μg | 4 μg | 5 μg | 6 μg |
| tration | | | | | | | | | | | | | | | |
| O.D. % | 100 | 150 | 99 | 103 | 111 | 140 | 154 | 153 | 162 | 173 | 185 | 190 | 189 | 191 | 190 |
| Standard | 12 | 10.75 | 10 | 11 | 12 | 10 | 12 | 10 | 12 | 12 | 12 | 12 | 12 | 11 | 12 |
| deviation | | | | | | | | | | | | | | | |

The cell survival rate in the group treated with bFGF increased 1.5 times, and that in the group treated with berberine was somewhat increased at concentrations of 250˜500 ng/ml, and the protective effect against apoptosis increased two times.

Although the protective effect against apoptosis did not further increase, the effect did not drop. Therefore, it is believed that there was no cytotoxicity. Berberine was determined to have a significant protective effect against apoptosis (p<0.05).

Therefore, it is believed that berberine has a protective effect against apoptosis during differentiation of adult neuronal stem cells and an effect of increasing cell survival.

### EXAMPLE 6

### Protective Effect of Berberine Against Apoptosis Induced by Stress in Differentiated Nerve Cell Line

### 1) Survival of Neuroblastoma Under Condition of Serum Deprivation-Induced Stress

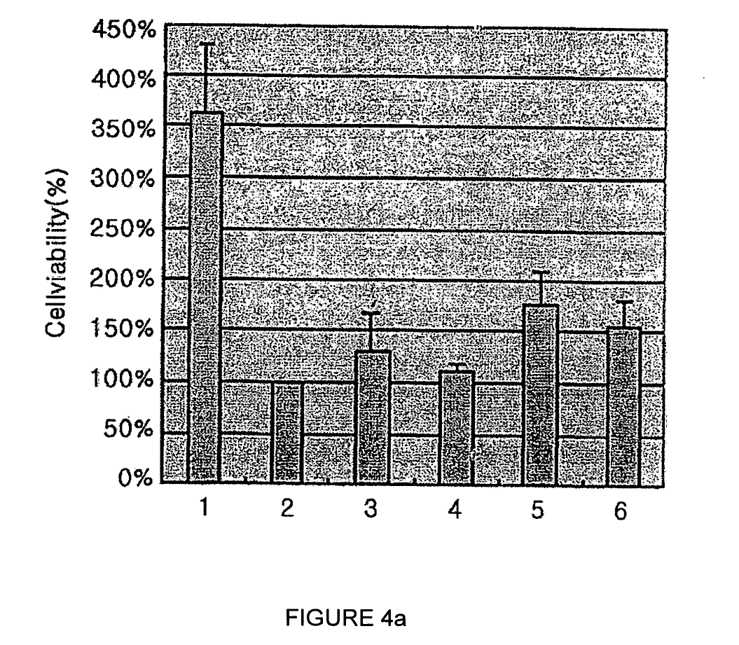

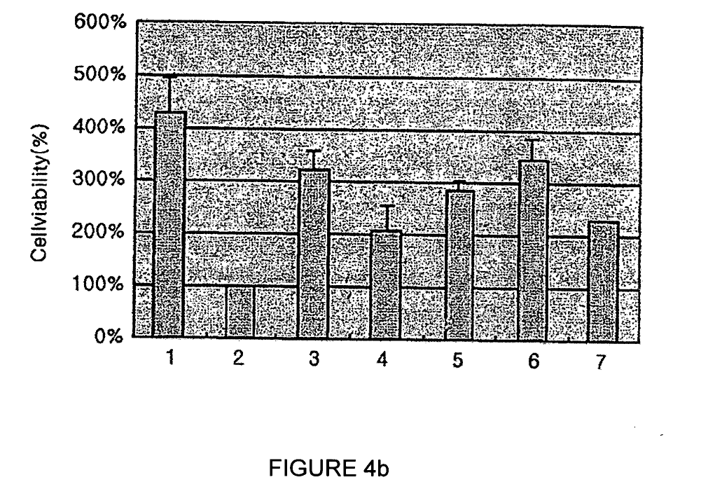

In order to prepare a stroke-like cell model using SH-SY5Y, the cells were cultured under serum deprivation. After the culture was treated with berberine, cell survival was evaluated by performing the MTT assay, thereby identifying the protective effect against cell injuries. Retinoic acid, which induces the differentiation of SH-SY5Y as a cell differentiation factor, was used as a positive control. 3 hours before depriving serum, the cells were treated with berberine (0.25˜3 μg). The cells were cultured in chemically defined media (N2) for 2 days to induce nerve regeneration (see, Table 2 and FIG. 4a).

TABLE 2

| | O.D. | Average | STE |
|---|---|---|---|
| Serum | 0.242 | 362% | 0.66911 |
| N2 | 0.067 | 100% | 0.00000 |
| N2 + Retinoic acid 5 μM | 0.871. | 130% | 0.36967 |
| | 87 | | |
| | 1. | | |
| N2 + Berberine 0.25 μg/ml | 0.076 | 113% | 0.05630 |
| N2 + Berberine 1.5 μg/ml | 0.118 | 177% | 0.33130 |
| N2 + Berberine 3 μg/ml | 0.104 | 155% | 0.27035 |
| P value (0.0247663) < 0.05 | | | |

The cell survival rate in the group treated with berberine (1.5 μg/ml) was two times higher than in the control group, but when treated with berberine (3 μg/ml), the cell survival was somewhat decreased. Therefore, it can be seen that berberine has an excellent protective effect against apoptosis, a positive effect on cell survival and a neuroprotective effect

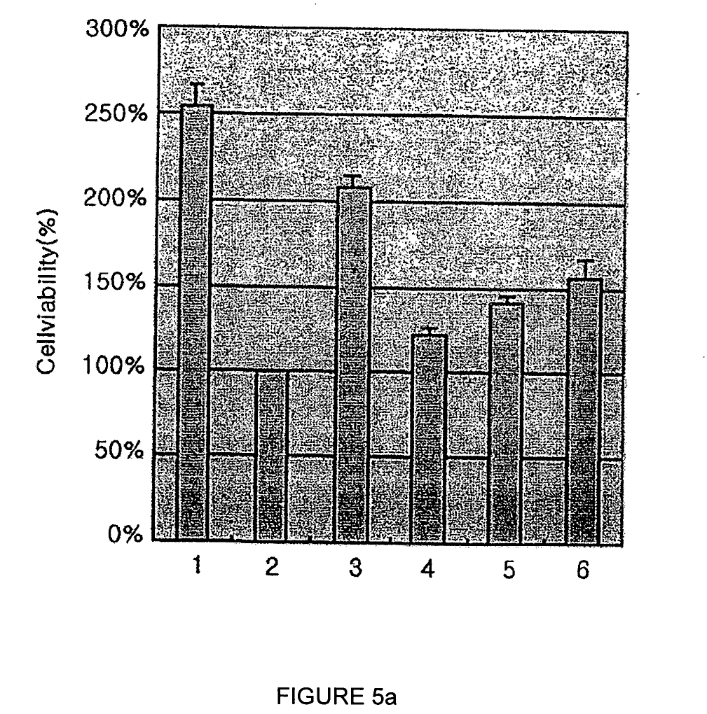

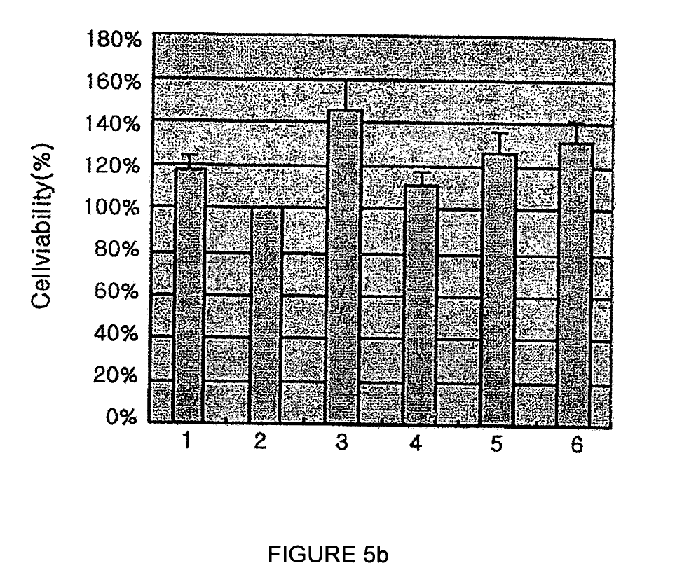

In addition, after adrenal tumor-derived rat PC 12 cells were treated with berberine under condition of serum deprivation-induced stress, the protective effect of berberine against cell injuries was examined. NGF, increasing cell survival and inducing cell differentiation, was used as a positive control. After the cells were treated with berberine (0.25˜3 μg/ml), the cells were cultured for 2 days to induce nerve regeneration (see, Table 3 and FIG. 5a).

TABLE 3

| | O.D. | Average | STE |
|---|---|---|---|
| Serum | 0.427 | 254% | 0.11109 |
| RPMI | 0.168 | 100% | 0.00000 |
| RPMI + NGF 100 ng/ml | 0.349 | 208% | 0.06587 |
| RPMI + Berberine 0.25 μg/ml | 0.203 | 121% | 0.04882 |
| RPMI + Berberine 1.5 μg/ml | 0.239 | 142% | 0.03996 |
| RPMI + Berberine 3 μg/ml | 0.262 | 156% | 0.10082 |

The cell survival rate in the group treated with berberine (1.5˜3 μg/ml) was 1.5 times higher than in the control group. Therefore, it is believed that berberine has an excellent neuroprotective effect in PC12 cells.

### 2) Survival of SH-SY Cells Under Condition of Oxidative Stress

SH-SY5Y cells were treated with H₂O₂ for 30 minutes to induce cell injuries. 3 hours before the cells were treated with H₂O₂, berberine (0.25˜3.5 μg) was added to cell culture to examine its cell protection effect (see, Table 4 and FIG. 4b).

TABLE 4

| | O.D. | Average | STE |
|---|---|---|---|
| N2 | 0.239 | 429% | 0.63867 |
| N2, H₂O₂ 150 μM | 0.056 | 100% | 0.00000 |
| N2, H₂O₂ + Retinoic acid 5 μM | 0.179 | 321% | 0.37410 |
| N2, H₂O₂ + Berberine 0.25 μg/ml | 0.116 | 208% | 0.46921 |
| N2, H₂O₂ + Berberine 1.5 μg/ml | 0.16 | 287% | 0.16892 |
| N2, H₂O₂ + Berberme 3 μg/ml | 0.192 | 344% | 0.37556 |
| N2, H₂O₂ + Berberine 3.5 μg/ml | 0.127 | 228% | 0.00000 |
| P value (0.043974) < 0.05 | | | |

As a result, the cell protection effect against oxidative stress in the group treated with berberine (3.0 μg/ml) was more than 3 times higher than in the control group. Therefore, it can be seen that berberine has an excellent protective effect against apoptosis of neuroblastomas and a positive effect on cell survival under condition of oxidative stress.

### 3) Effect of Berberine on Survival of Neuroblastoma Cells Under Condition of Glucocorticoid Stress

Glucocorticoids are hormones secreted from the adrenal glands, and involve in a variety of metabolic processes including glycometabolism. In particular, stress causes release of glucocorticoids to reduce cell divisions of neuronal stem cells in the brain and induce apoptosis of nerve cells. Therefore, PC12 cells were treated with dexamethasone, a glucocorticoid derivative, to examine the effect of berberine against apoptosis of nerve cells. 3 hours after treatment with berberine, dexamethasone (1) was added to a culture solution and incubated for 2 days to induce neuroregeneration (see, Table 5 and FIG. 5b).

TABLE 5

| | O.D. | Average | STE |
|---|---|---|---|
| N2 | 0.227 | 118% | 0.06300 |
| N2 + Dexamethasone 1 μM | 0.193 | 100% | 0.00000 |
| N2 + Dexamethasone + NGF 100 ng/ml | 0.283 | 147% | 0.13536 |
| N2 + Dexamethasone + Berberme 0.25 μg/ml | 0.214 | 111% | 0.07494 |
| N2 + Dexamethasone + Berberine 1.5 μg/ml | 0.245 | 127% | 0.09335 |
| N2 + Dexamethasone + Berberme 3 μg/ml | 0.253 | 132% | 0.09528 |

The cell survival rate in the group treated with NGF as a positive control increased about 1.5 times, whereas the cell survival rate in the group treated with berberine increased about 1.3 times. Therefore, it is believed that berberine can increase survival of PC12 cells under condition of dexamethasone stress.

### EXAMPLE 7

### Regenerative Effect of Berberine on Differentiation of Nerve Cells

### 1) Induction of Differentiation

In order to identify the effect of berberine on inducing differentiation of neuronal stem cells, HiB5 cells were cultured under conditions for initiation of differentiation for 1 day. Thereafter, the culture was treated with berberine and further cultured for 2 days. The cultured cells were immunostained with nerve cell-specific labeled molecule, and then the number of cells having neurites longer than cell bodies was counted under a confocal microscope. A positive control was treated with bFGF under the same condition as described above to induce the differentiation into nerve cells. In addition, the neuroregenerative effect was examined after berberine together with bFGF was treated. The differentiation degree was measured by double-staining neurites with nerve cell-specific labeled molecule (anti-neurofilament antibody) and FITC-labeled secondary antibody (green), followed by staining cell nuclei with propidium iodide (red).

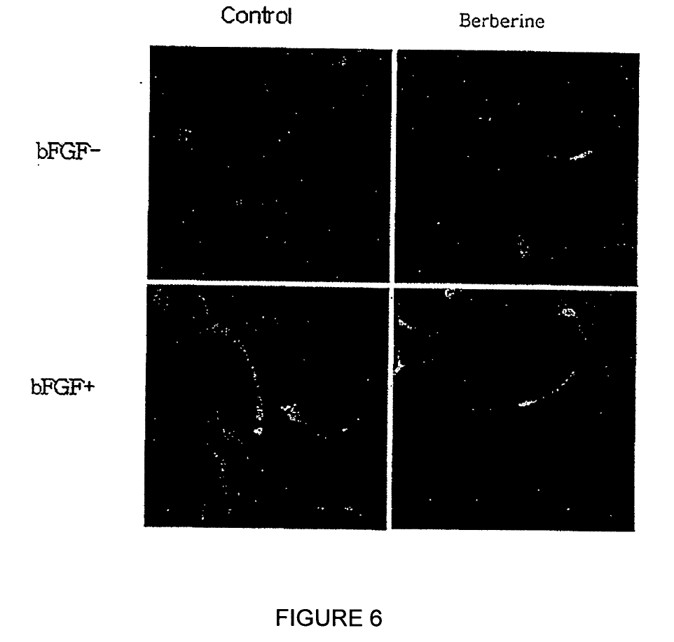

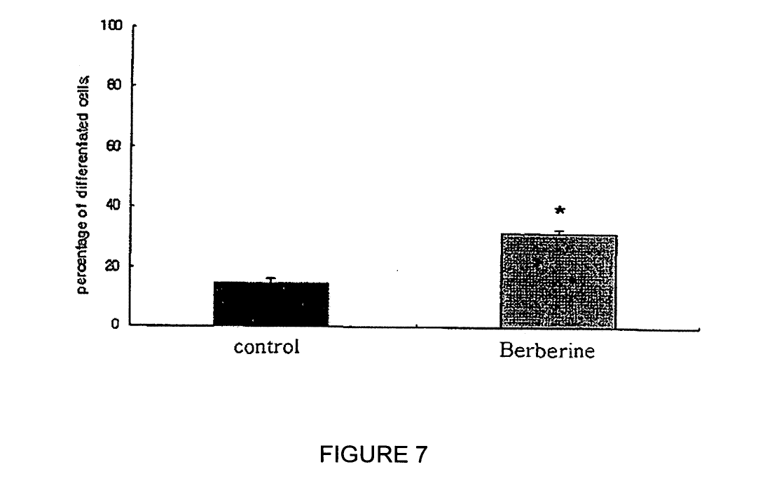

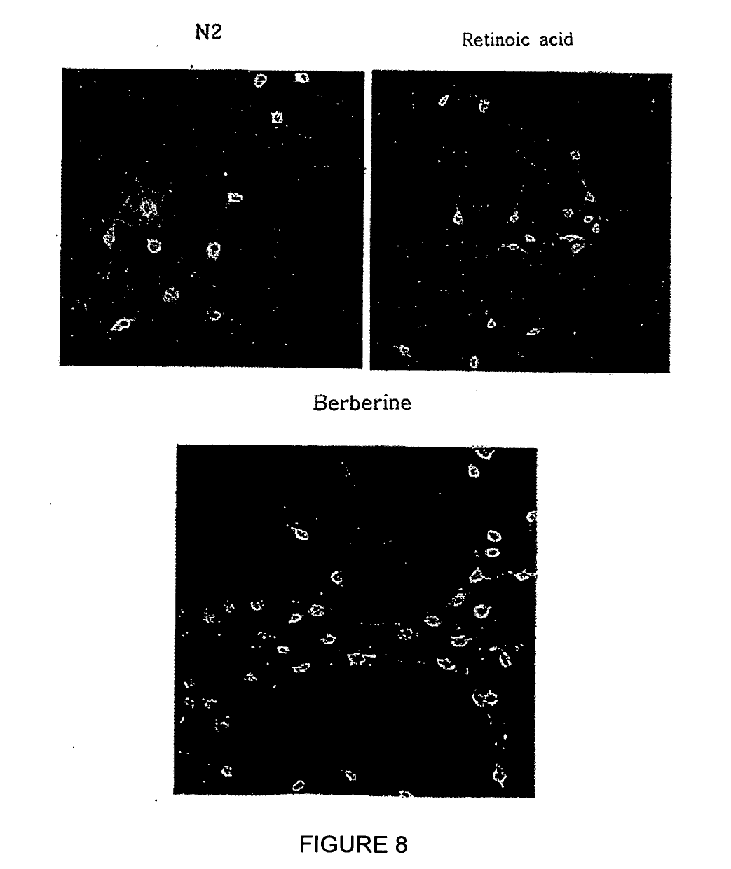

As shown in FIG. 6, treatment with bFGF induced the differentiation into nerve cells. At this time, cell bodies got smaller and neurites got longer. The group treated with berberine increased two times in the number of cells differentiated into nerve cells. Therefore, it can be seen that berberine can induce the differentiation into nerve cells (see, Table 6 and FIG. 7).

TABLE 6

| | N2 | Berberine |
|---|---|---|
| Number of differentiated cells/ | 3.07/22.38 | 7.87/26.61 |
| total average cell numbers | | |
| Average (%) | 14.46 | 31.57 |

### 2) Effect on Neurite Regeneration

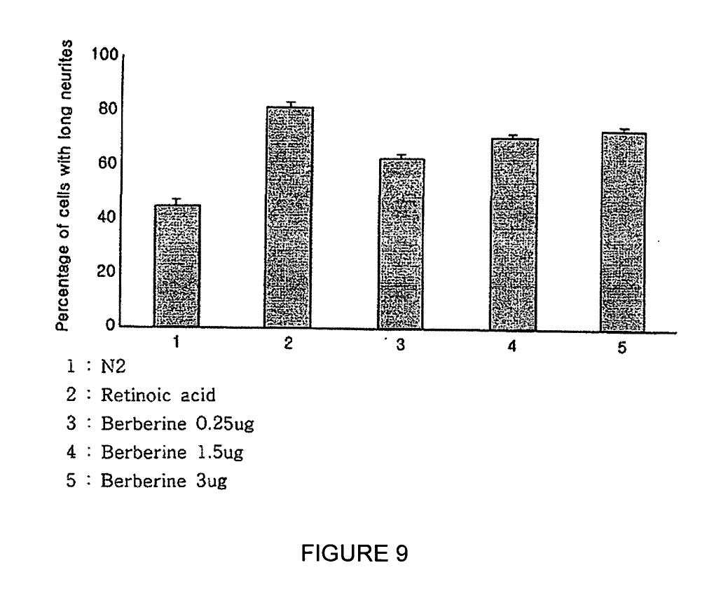

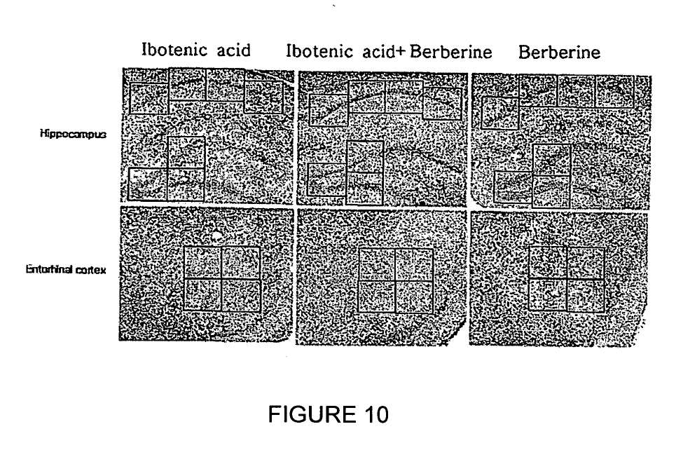

In order to examine the effect of berberine on neurite regeneration, SH-SY5Y cells were used as differentiated nerve cell lines. Retinoic acid inducing neurite growth was used as a positive control. It was observed that berberine in SH-SY5Y cells increased two times in the number of cells and lengthened the length of neurites. In addition, the number of cells having neurites three times longer than cell bodies increased 1.7 times (see, Table 7 and FIG. 9).

TABLE 7

| | | | Berberine | Berberine | Berberine |
|---|---|---|---|---|---|
| | N2 | Retinoic acid | 0.25 μg/ml | 1.5 μg/ml | 3 μg/ml |
| Number of | 22.30/42.33 | 18.91/24.75 | 26.51/39.75 | 27.89/38.80 | 28.64/38.60 |
| differentiated cells/ | | | | | |
| total average cell | | | | | |
| numbers | | | | | |
| Average (%) | 44.47 | 81.59 | 63.34 | 71.04 | 73.95 |

### EXAMPLE 8

### Neuroregenerative Effect of Berberine in Animal Model for Dementia

As an animal model for dementia, ibotenic acid was microinjected into the entorhinal cortex of adult rat brain using a stereotaxic frame. Since injection of ibotenic acid into the entorhinal cortex exhibiting serious degeneration kills pyramidal cells in the CA1 region and granule cells in the dentate gyrus regions, the animal model for dementia is also used a model for chemical lesion.

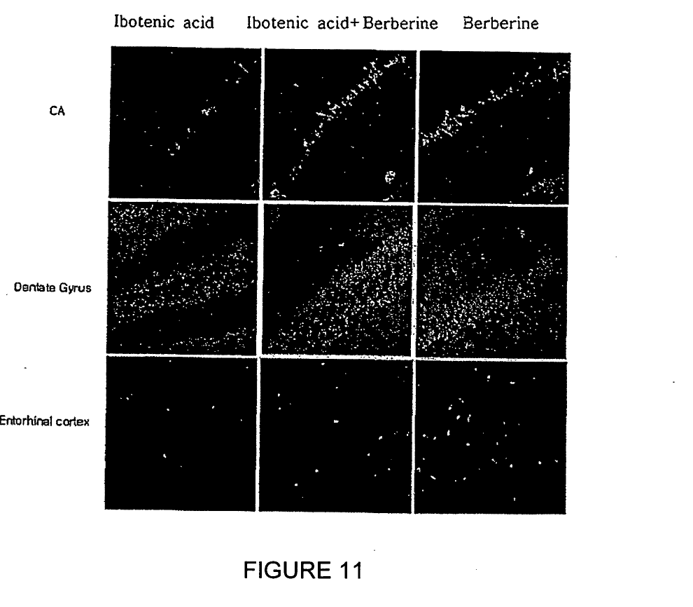

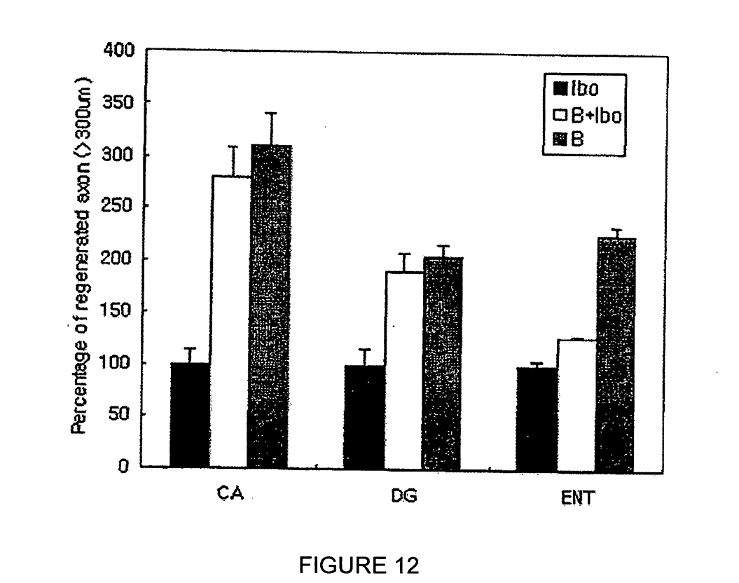

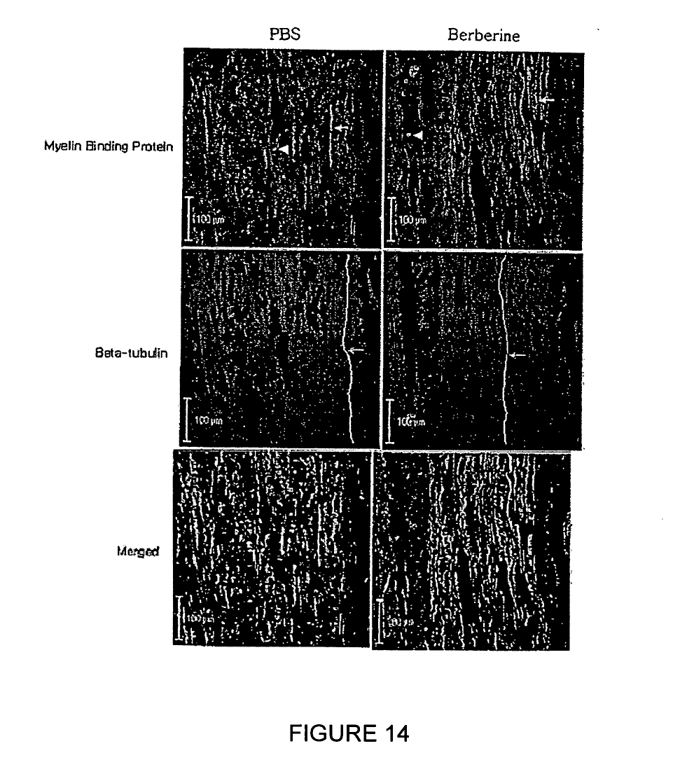

First, an adult rat was anesthetized, and its head was fixed with a stereotaxic frame. Subsequently, 1.5 μl (1 mg/ml) of ibotenic acid was injected into the entorhinal cortex region to prepare a model for dementia, and then berberine was injected into the same site. 2 weeks after operation, brain tissue sections were counter-stained with hematocylin to examine apoptosis and protective effect. After fluorescence-staining the cells, regions used for cell count were represented as squares (FIG. 14). In addition, the cells were fluorescence-stained with calbindin antibody, a nerve-specific marker, to examine nerve cell survival (see, Tables 8 to 10, FIGS. 11 and 12).

TABLE 8

| | | DG | ENT | | | |
|---|---|---|---|---|---|---|
| | | (Dentate | (Entorhinal | STDE/ | STDE/ | STDE/ |
| (%) | CA | Gyrus) | Cortex) | CA | DG | ENT |
| Ibotenic | 100 | 100 | 100 | 13.68 | 14.89 | 3.17 |
| Acid | | | | | | |
| Ibotenic | 278 | 190 | 126 | 31.11 | 18.47 | 2.07 |
| Acid + | | | | | | |
| Berberine | | | | | | |
| Berberine | 311 | 204 | 225 | 28.63 | 12.41 | 8.6 |

TABLE 9

| Ibotenic Acid + | | | |
|---|---|---|---|
| Berberine | CA | DG (Dentate Gyrus) | ENT (Entorbinal Cortex) |
| #1 | 113 | 406 | 94 |
| #2 | 165 | 289 | 89 |
| #3 | 131 | 351 | 93 |
| AVR | 136.3 | 348.7 | 92 |
| STD | 26.4 | 58.5 | 2.6 |
| STDE | 15.2 | 33.7 | 1.5 |
| Berberine | CA | DG | ENT |
| #1 | 174 | 421 | 173 |
| #2 | 126 | 347 | 152 |
| #3 | 157 | 358 | 167 |
| AVR | 152.3 | 375.3 | 164 |
| STD | 24.3 | 39.9 | 10.8 |
| STDE | 14 | 23 | 6.2 |
| Ibotenic Acid | CA | DG | ENT |
| #1 | 57 | 227 | 73 |
| #2 | 55 | 193 | 77 |
| #3 | 36 | 133 | 68 |
| AVR | 49.3 | 184.3 | 72.7 |
| STD | 11.6 | 47.6 | 4.5 |
| STDE | 6.6 | 27.4 | 2.5 |

| P value | |
|---|---|
| CA | 0.032557 |
| DG | 0.04473 |
| ENT | 0.037336 |

TABLE 10

| Ibotenic Acid + | | | |
|---|---|---|---|
| Berberine | CA | DG (Dentate Gyrus) | ENT (Entorbinal Cortex) |
| #1 | 231 | 221 | 129 |
| #2 | 337 | 157 | 122 |
| #3 | 267 | 191 | 127 |
| AVR | 278 | 190 | 126 |
| STD | 53.9 | 32.0 | 3.6 |
| STDE | 31.1 | 18.5 | 2.1 |
| Berberine | CA | DG | ENT |
| #1 | 355 | 229 | 237 |
| #2 | 257 | 189 | 208 |
| #3 | 320 | 195 | 229 |
| AVR | 311 | 204 | 225 |
| STD | 49.7 | 21.6 | 15 |
| STDE | 28.6 | 12.4 | 8.6 |
| Ibotenic Acid | CA | DG | ENT |
| #1 | 116 | 123 | 100 |
| #2 | 112 | 105 | 105 |
| #3 | 73 | 72 | 94 |
| AVR | 100 | 100 | 100 |
| STD | 23.8 | 25.9 | 5.5 |
| STDE | 13.7 | 14.9 | 3.2 |

| P value | |
|---|---|
| CA | 0.032218 |
| DG | 0.045397 |
| ENT | 0.033969 |

As shown in FIG. 11, 2 weeks after injecting ibotenic acid alone, the number of calbindin-positive neurons was reduced by 30˜40% in the hippocampus and entorhinal cortex, and 4 weeks after injection, the cell number recovered somewhat. Cell numbers in the dentate gyrus regions were reduced more slowly than cell numbers in the CA1 region. However, in the case of injecting berberine, survival of calbindin-positive neurons increased, in particular, survival rates of pyramidal cells in the CA1 region increased 2.5 times, and those of granule cells in the dentate gyrus regions doubled. Also, survival of calbindin-positive neurons in the entorhinal cortex were somewhat increased, but the increase was statistically significant (FIG. 12, P<0.034). Therefore, it is believed that berberine has a protective effect against apoptosis of nerve cells and a neuroregenerative effect in an animal model for dementia.

### EXAMPLE 9

### Effect of Berberine on Regeneration of Sciatic Nerves in the Peripheral Nervous System

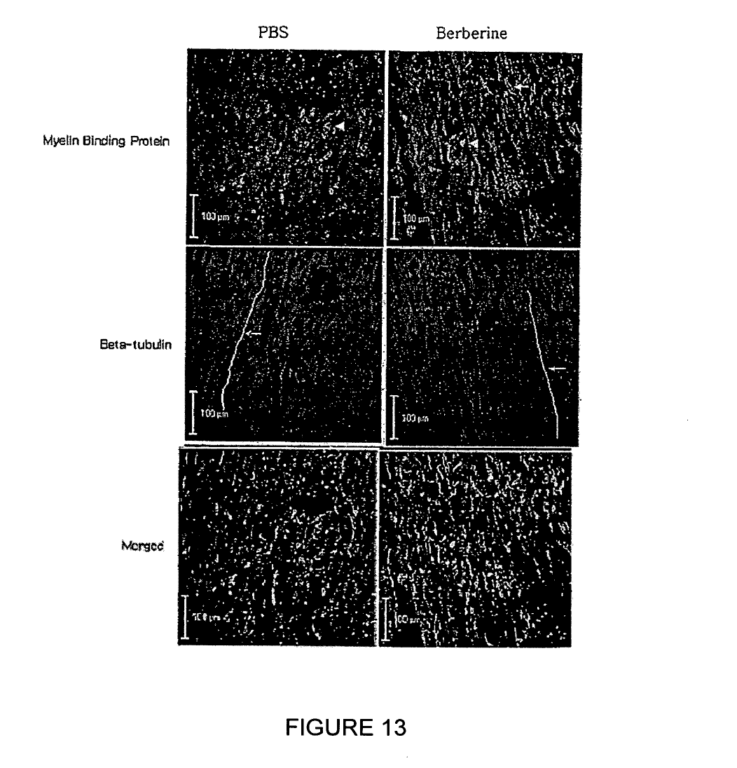

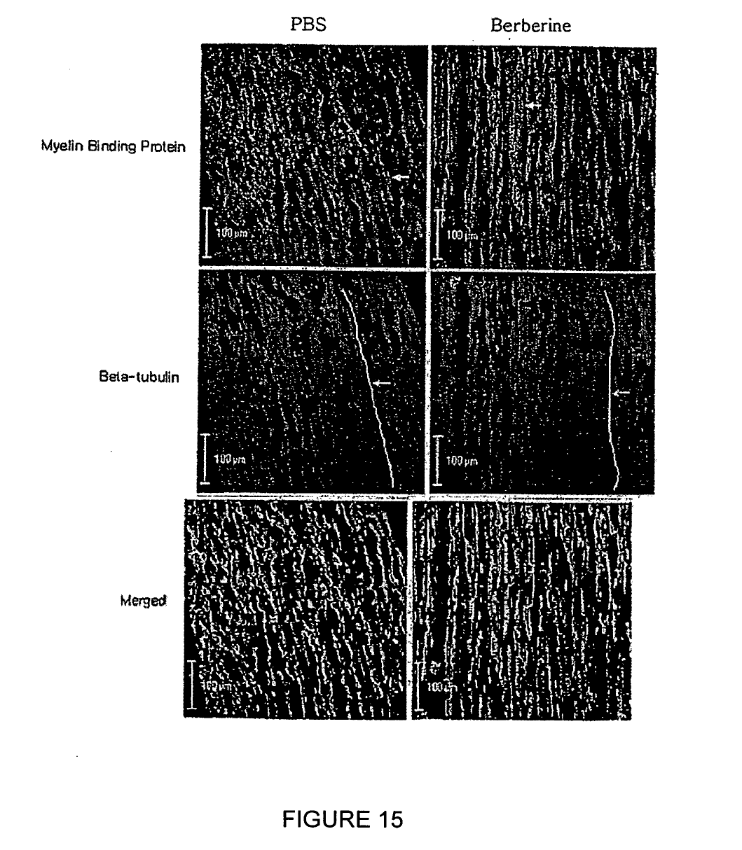

After a white rat was anesthetized, its sciatic nerves were exposed and crushed. PBS or berberine was intraperitoneally injected into the rat. 1 week, 2 weeks and 4 weeks after suturing, nerve regeneration was observed. The rat was perfused and then sciatic nerves were obtained from the distal stump. After the obtained sciatic nerves were cryosected to a thickness of 7-10 μm, cryosected sciatic nerves were double-stained using beta-tubulin isotypeIII (cy3, red), which is an axon marker, and MBP (myelin binding protein, cy2, green) antibody, which is a differentiation (myelin) marker of Schwann cells. It was observed under a confocal microscope that axons (see, Table 11) were longer than 300 μm and myelin sheaths (see, Table 12) were longer than 200 μm (see, FIGS. 13 to 15, 19a and 19b).

TABLE 11

| | PBS | Berberine | STDE/PBS | STDE/Berberine |
|---|---|---|---|---|
| 1 week | 14.5 | 24.6 | 3.493 | 2.026 |
| 2 weeks | 20.5 | 32.3 | 2.496 | 6.356 |
| 3 weeks | 21.3 | 51.6 | 2.401 | 10.16 |

TABLE 12

| | PBS | Berberine | STDE/PBS | STDE/Berberine |
|---|---|---|---|---|
| 1 week | 16.5 | 35 | 0.49 | 1.732 |
| 2 weeks | 43 | 88.6 | 2.998 | 15.67 |
| 3 weeks | 40 | 112 | 12.48 | 10.32 |

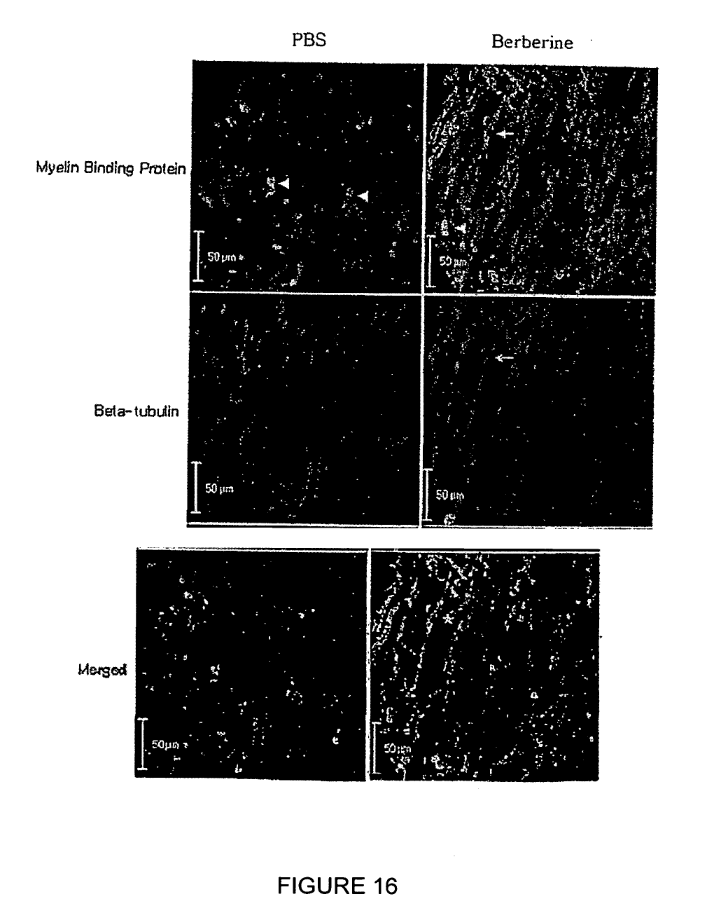

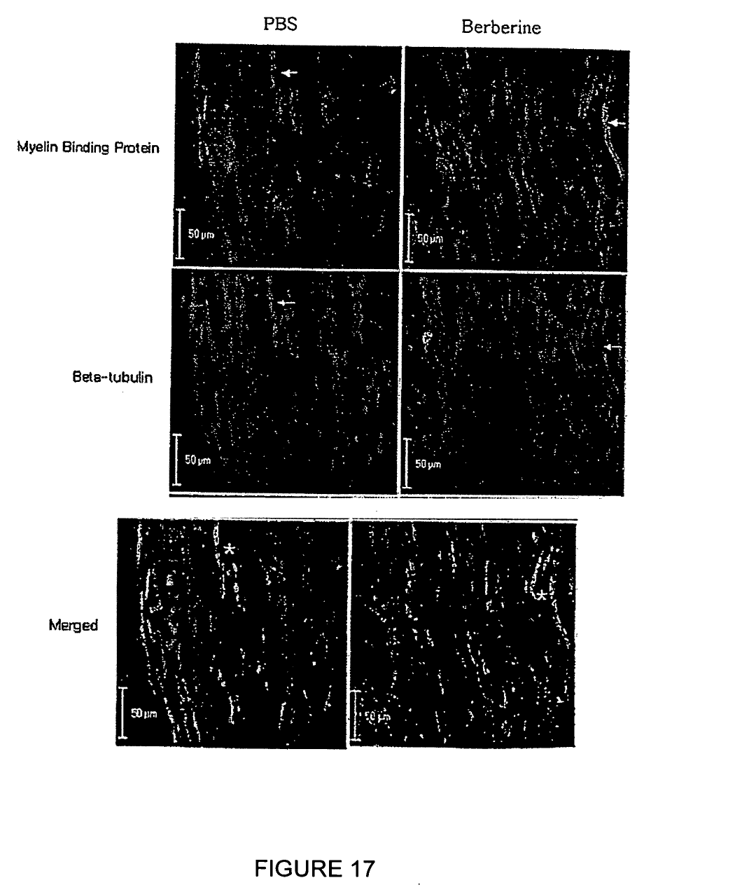

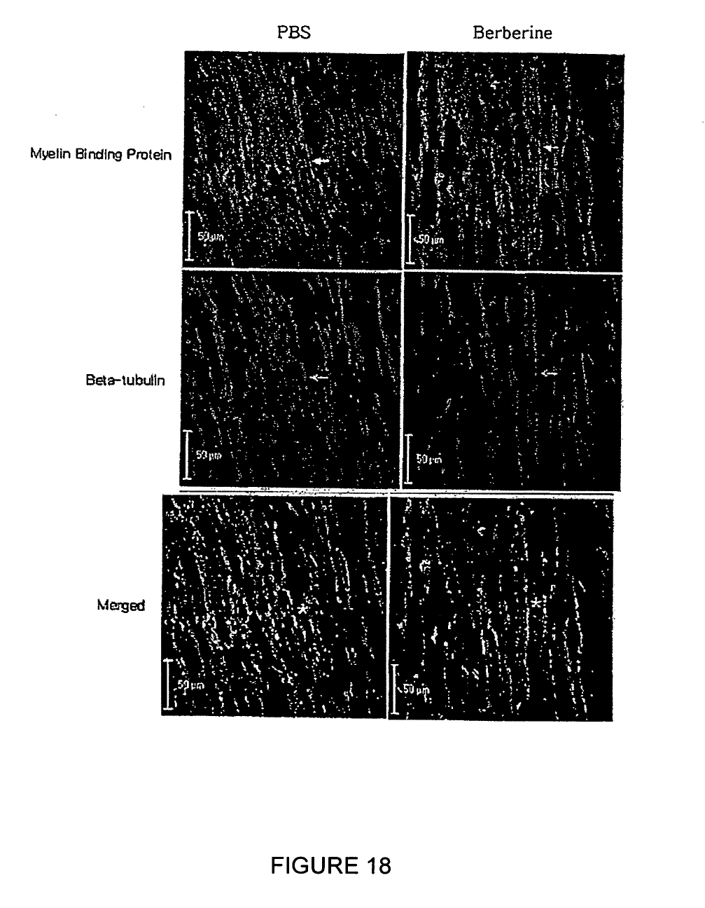

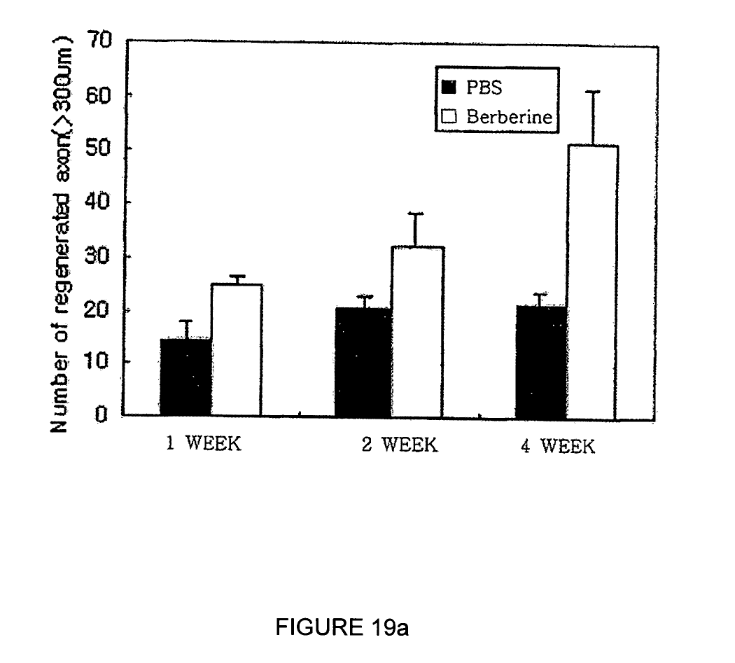

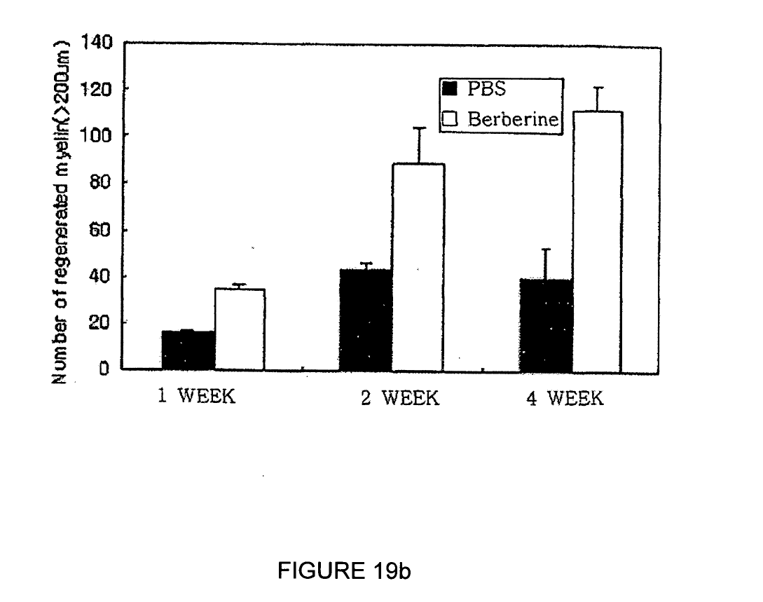

1 week after operation, since axons and myelin sheaths underwent degeneration at the distal stumps, degenerating myelin sheaths were stained (arrows in FIGS. 13 and 16). Effect of berberine on axonal growth and the regeneration of myelin sheaths was not great, but 4 weeks after operation, the number of axons longer than 300 μm had doubled (white lines). 1 week after operation, the number of myelin sheaths longer than 200 μm had doubled, and 4 weeks after operation, the number had increased by 3 times (see, Table 11, FIGS. 19a and 19b).

Therefore, berberine promotes axonal growth and the regeneration of myelin sheaths during regeneration of peripheral nerves.

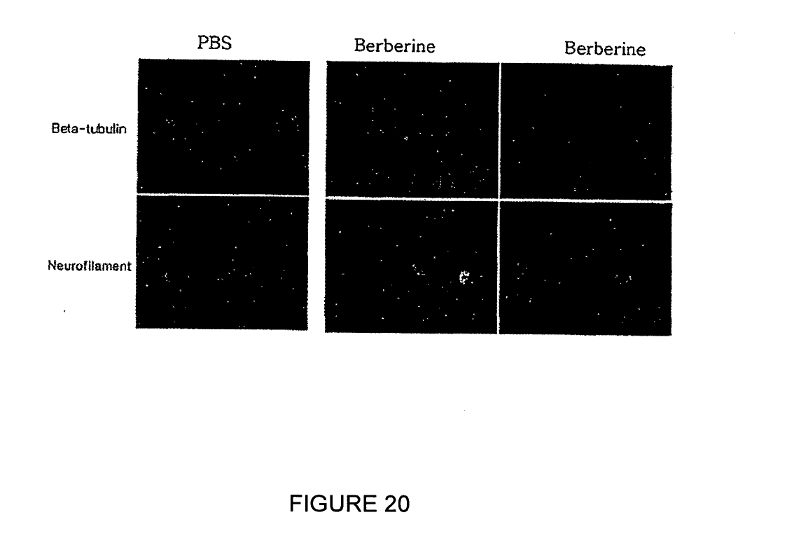

In order to see whether berberine influences the regeneration of nerve endings in the neuromuscular junctions, 4 weeks after operation, hindlimb muscles connected to sciatic nerves were separated and cryosected. The neuromuscular junctions were stained using beta-tubulin isotypeIII and neurofilament, which are nerve markers. 4 weeks after operation, it was observed in a control group that nerve endings were stained, but did not spread to muscle fibers and thus did not form neuromuscular junctions. However, in the group administered with berberine, the nerve endings spread to all muscle fibers (FIG. 20).

Therefore, berberine promotes axonal growth, the regeneration of myelin sheaths and the regeneration of nerve endings to form neuromuscular junctions during regeneration of peripheral nerves.

### EXAMPLE 10

### Neuroregenerative Effect of Berberine in Animal Model for Stroke

### 1) Methods and Materials

### (1) Forebrain Ischemia Induced by 4-Vessel Occlusion (4-VO)

A 4-VO model was prepared by a modified method of Pulsinelli, et al (Ann. Neurol. 11, 491-498(1982)). Wister male white rats weighing about 18˜200 g were anesthetized with 5% isoflurane contained in a mixture of nitrogen and oxygen (70:30), and then surgery was performed under anesthetization while maintaining the concentration of isoflurane at 1.5%.

First, the throat region was incised, and then silicone tube rings were inserted into common carotid arteries to perform reperfusion after ischemia induction. Upon inducing ischemia, in order to block blood circulation through microvessels, a thread was penetrated so that the trachea, esophagus, external jugular veins and common carotid arteries of the rat were positioned to the front, and cervical and paravertebral muscles of the rat were positioned to the back. Thereafter, the wounds were sutured with operating clips.

Next, the head of the rat was fixed on a stereotaxic apparatus to operate on the occiput, and then the tail was fixed so that it descended downwardly at an angle of 30°. After incising the occipital bone, an electrocauterizing needle having a diameter of 1 mm or less was inserted into the alar foramina positioned at lower part of the first cervical vertebra under the occipital bone. At this time, this approach must be carefully done so as not to damage the muscles in the alar foramina. Thereafter, the vertebral artery was electrically cauterized by intermittently applying current. After the complete electrocauterization of the vertebral artery was confirmed, suturing was carried out using operating clips. After 24 hours, the operating clips were removed. Finally, the common carotid arteries were occluded using the silicone tube rings for 10 minutes to induce ischemia. If light reflex did not disappear within 1 minute, the cervical portion was further tightly sutured. Rats which did not show the complete disappearance of light reflex were excluded from the experiment because they underwent no damage to the CA1 region. After 10 minutes, the common carotid arteries were loosened to reperfuse. For 20 minutes after the reperfusion, loss of consciousness was observed. At this time, only rats which showed consciousness loss period within 20±5 minutes were selected for subsequent experiments.

While body temperatures of the rats were measured at intervals of 30 minutes for 2 hours, and thereafter measured at intervals of 1 hour for 3 hours after ischemia induction, the rats were maintained at 37±0.5° C. using a temperature-controlling heater. The body temperature was measured by inserting a probe into the rectum to a depth of 6 cm. The rectal temperature reflects brain temperature.

### (2) Sample Administration and Selection of Experiment Group

In order to evaluate efficiency of berberine against forebrain ischemia in white rats, berberine was intraperioneally administered the rats in a single dose. 0 and 90 minutes after forebrain ischemia induction, berberine was administered.

Experimental animals were randomly divided into the following 3 groups: the first group (a normal control group) underwent an operation in the same manner, but forebrain ischemia was not induced; the second group (a control group) was intraperitoneally administered with physiological saline (2.0 ml/kg) at the same time intervals as the sample administration after forebrain ischemia induction; and the third group was intraperitoneally administered with berberine in a single dose 0 and 90 minutes after forebrain ischemia induction.

### (3) Preparation of Tissue Sections

For histological examination, 1 week after forebrain ischemia induction, the rat was anesthetized with chloral hydrate ( [symbol] , Japan, 35.0 mg/kg, i.p.) and its chest was opened. The right auricle was incised and a needle (No. 18) was inserted into the left ventricle. Thereafter, the heart was perfused with heparinized, 0.5% sodium nitrite (Sigma, U.S.A.) physiological saline, and further perfused with 4.0% formalin fixative (PFA, Sigma, U.S.A) dissolved in 0.1M phosphate buffer having a pH of 7.4. After the brain was removed from the rat and fixed with 4% paraformaldehyde at a temperature of 4° C. for 2 hours, the brain was washed with 0.1M phosphate buffer twice, immersed in 30% sucrose (Sigma, U.S.A.), and then stored at 4° C. overnight. A coronal block in the dorsal hippocampus portion between −2.5 mm and −4.0 mm from the bregma was prepared. After the coronal block thus prepared was frozen, tissue sections including the hippocampus were prepared using a sliding microtome (HM440E, Zeiss, Germany). Tissue sections were collected every 30 μm.

### (4) Observation of Number of Damaged Nerve Cells

After the tissue sections including the dorsal hippocampus were stained with cresyl violet and fixed, nerve cells in the 1,0001 μm long central area, which is the most susceptible to delayed neuronal death in the CA1 of the dordal hippocampus, were counted. The number of nerve cells was counted by averaging pyramidal cell numbers having normal morphology in the left and right sides (total 6 sites) of three different tissue sections counted by three observers. The counting was performed under a microscope (×400). At this time, all observers were not informed which samples belonged to experimental or control subjects.

### (5) Statistics

In order to determine all effects of berberine, each experimental group was compared with the control group using Student's t-test.

### 2) Experimental Results

### (1) Concentration of Berberine, Influence of Body Temperature and Ischemia Inducing Time

The highest concentration of berberine was set to 300 μg/0.1 kg, and 600 μl (1 mg/ml) of berberine was intraperitoneally injected to white rats weighing 200 g. In order to determine an optimal ischemia induction time, 2˜3 rats were selected and ischemia-induced over 5, 10, 20 and 30 minutes, respectively. 1 week after reperfusion, they were sacrificed and their hippocampal tissue sections were obtained to observe the number of damaged nerve cells. 10 minutes after ischemia induction, damaged pyramidal cells in the hippocampal CA1 region were found to be reduced to ¼ of their original numbers. The ischemia induction time of 10 minutes was determined to be most optimal for evaluating the effects of berberine.

For statistically analyzing the effects of berberine, a sham operated group having undergone an operation in the same manner without ischemia induction was used. For comparing the effects of berberine, a control group administered with physiological saline at the same dose as berberine was used. Berberine was intraperitoneally injected into all experimental groups.

It is well known that reduction in body temperature during ischemia induction prevents damage to nerve cells in the hippocampus and thus exhibits neuroprotective effects. Therefore, in order to evaluate the neuroprotective effect of berberine, after ischemia induction and reperfusion, the body temperature of all rats was maintained at a constant (37±1° C.) for 6 hours.

### (2) Observation of Damaged Nerve Cells

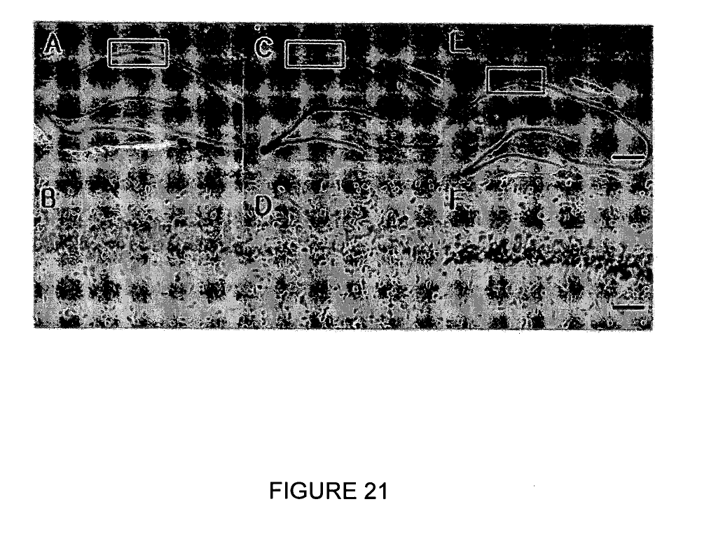

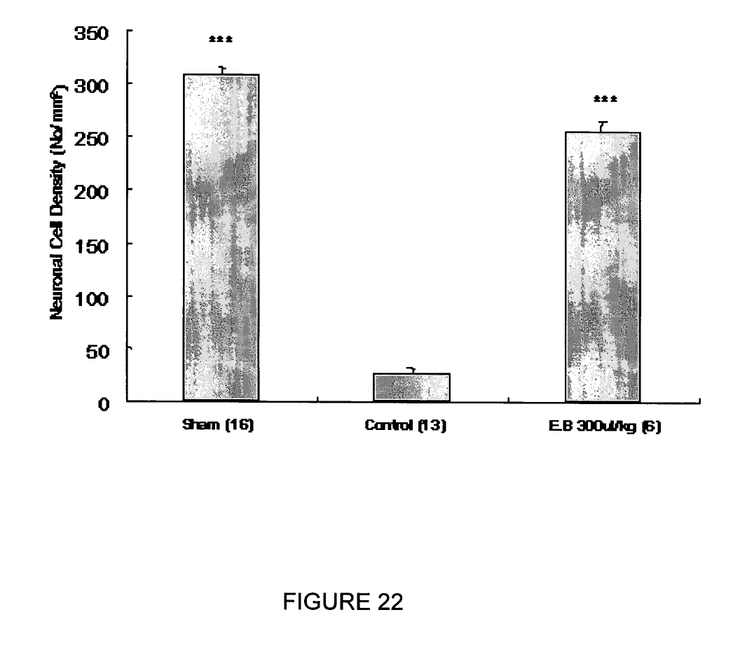

When ischemia was induced by 4-VO and then reperfusion was performed, nerve cells in the neocortex, striatum, hippocampal CA1 region and cerebellum were damaged. Among them, pyramidal nerve cells in the hippocampal CA1 region were the most susceptible to the induced ischemia, and started to undergo cell death 72 hours after reperfusion. In order to observe delayed neuronal death in the hippocampal CA1 region, 1 week after reperfusion, the time when almost all nerve cells were damaged, white rats were sacrificed and tissue sections from the hippocampus were observed under an optical microscope. In a sham operated group having undergone no ischemia, normal hippocampal nerve cells were observed in the stratum pyramidale (490 μm long)(see,A and B of FIG. 21).

C and D of FIG. 21 as control groups show apoptosis. When cells are induced to undergo apoptosis by an external or an internal stimulus, they shrink to lose their original shapes. This shrinkage breaks the junctions with other adjacent cells so that the interaction between cells is disrupted. When the shrinkage proceeds to some extent, the cell membranes form apoptotic bodies like a bulla. In the hippocampal CA1 region of the control group administered with physiological saline (D of FIG. 21), it was observed that nerve cells underwent apoptotic morphological changes after ischemia induction. In addition, it was observed that tissues was relaxed and separated from adjacent cells, unlike B of FIG. 21. From these observations, it was confirmed that the cell bodies of nerve cells lost their original pyramidal shape and were condensed, thereby appearing to be single cells. Furthermore, it was confirmed that subsequent nuclear chromatin condensation and nuclear envelope collapse led to apoptosis of nerve cells. On the contrary, nerve cells in the hippocampal CA1 region administered with berberine were similar to normal cells in terms of their morphology (see, E and F of FIG. 21). At this time, because necrotic nerve cells around the CA1 region were very difficult to distinguish from microglias, only viable pyramidal nerve cells in the CA1 region were counted. In F of FIG. 21, separated cells were observed above and below the hippocampal region and cell bodies were condensed. This demonstrates that the damage to nerve cells was great enough to induce apoptosis. Nevertheless, it was observed that a great number of nerve cells were protected from apoptosis and their original pyramidal morphology was maintained. This suggests that berberine has a protective effect against damages to nerve cells in the hippocampal CA1 region induced by 4-VO. Although it was not confirmed what stage during apoptosis influences nerve cell survival, it was certain that berberine has a significant protective effect against apoptosis of nerve cells (see, E and F of FIG. 21).

### (3) Protective Effect of Berberine Against Damage to Nerve Cells

In order to examine the neuroprotective effect of berberine after ischemia induction, berberine was intraperitoneally injected 0 and 90 minutes after ischemia induction.

In the sham groups, the density of viable cells was measured to be 308±6.6 cells/mm² (at 37° C.). In the control groups administered with physiological saline, the density of viable cells was measured to be 28±3.8 cells/mm² (at 37° C.). There was cell loss in these two groups. On the other hand, in the experimental groups administered with berberine, the density of viable cells was measured to be 257±9.6 cell/mm². In conclusion, berberine was determined to have a significant neuroprotective effect (p<0.05).

As described above, the composition according to the present invention regenerates axons and dendrites of nerve cells, thereby having a protective effect against nerve cell injuries, a positive effect on nerve cell growth and a regenerative effect on nerve cells. In addition, the composition according to the present invention can be used as a therapeutic agent for the prevention and treatment of neurodegenerative diseases or nerve injuries, in particular, dementia, Parkinson's disease, Alzheimer's disease, epilepsy, palsy, ischemic brain diseases, trauma to the spinal cord and peripheral nerve injuries.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

We claim:

1. A composition for protecting nerve cells, promoting nerve cell growth and regenerating nerve cells, or for preventing and treating nerve injuries or nervous diseases, comprising a compound of the following formula 1: 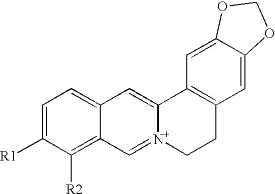
wherein
R1 and R2 are the same or different from each other and independently selected from the group consisting of alkoxy group, alkyl group, hydrogen atom, methylenedioxy group, substituted benzyl group, propoxy group, octyl group, alkenyl group, alkynyl group, amino group, amide group, cyano group, thiocyano group, aldehyde group and halogen atom,
derivatives thereof, or pharmaceutically acceptable salts thereof.

2. A composition for pretreating nerve cells with a compound of formula 1, derivatives thereof or pharmaceutically acceptable salts thereof in order to protect nerve cells, promote nerve cell growth and regenerate nerve cells, or to prevent and treat nerve injuries or nervous diseases.

3. The composition as set forth in claim 1, wherein R1 and R2 are methoxy group.

4. The composition as set forth in claim 1, wherein the pharmaceutically acceptable salt is berberine chloride.

5. The composition as set forth in claim 1, wherein the nerve cells are neuronal stem cells.

6. The composition as set forth in claim 1, wherein the nerve injuries and nervous diseases are brain injuries and brain diseases, respectively.

7. The composition as set forth in claim 6, wherein the brain injuries or brain diseases include dementia, Parkinson's disease, Alzheimer's disease, Huntington's disease, epileptic, palsy, stroke, ischemic brain diseases, degenerative brain diseases and memory loss.

8. The composition as set forth in claim 1, wherein the nerve injuries or nervous diseases include peripheral nerve injuries, neuromuscular disorders, amyotrophic lateral sclerosis and peripheral nervous diseases.

9. The composition according to claim 1, wherein the nerve injuries or nervous diseases include trauma to the spinal cord and the nervous system diseases related to the spinal cord.

10. The composition as set forth in claim 1, wherein the composition is used as foods or drugs.

11. The composition as set forth in claim 2, wherein R1 and R2 are methoxy group.

12. The composition as set forth in claim 2, wherein the pharmaceutically acceptable salt is berberine chloride.

13. The composition as set forth in claim 2, wherein the nerve cells are neuronal stem cells.

14. The composition as set forth in claim 2, wherein the nerve injuries and nervous diseases are brain injuries and brain diseases, respectively.

15. The composition as set forth in claim 14, wherein the brain injuries or brain diseases include dementia, Parkinson's disease, Alzheimer's disease, Huntington's disease, epileptic, palsy, stroke, ischemic brain diseases, degenerative brain diseases and memory loss.

16. The composition as set forth in claim 2, wherein the nerve injuries or nervous diseases include peripheral nerve injuries, neuromuscular disorders, amyotrophic lateral sclerosis and peripheral nervous diseases.

17. The composition according to claim 2, wherein the nerve injuries or nervous diseases include trauma to the spinal cord and the nervous system diseases related to the spinal cord.

18. The composition as set forth in claim 2, wherein the composition is used as foods or drugs.
